SEQUENCE LISTING (18 sequences)

SEQ ID NO: 1 - protein, 559 aa, Artificial Sequence
  YTH N6-Methyladenosine RNA Binding Protein I (YTHDFI)
MSATSVDTQRTKGQDNKVQNGSLHQKDTVHDNDFEPYLTGQSNQSNSYPSMSDPYLSSYYPPSIGFPYSLNEAPWSTAGDPPIPYLTTYGQLSNGDHHFMHDAVFGQPGGLGNNIYQHRFNFFPENPAFSAWGTSGSQGQQTQSSAYGSSYTYPPSSLGGTVVDGQPGFHSDTLSKAPGMNSLEQGMVGLKIGDVSSSAVKTVGSVVSSVALTGVLSGNGGTNVNMPVSKPTSWAAIASKPAKPQPKMKTKSGPVMGGGLPPPPIKHNMDIGTWDNKGPVPKAPVPQQAPSPQAAPQPQQVAQPLPAQPPALAQPQYQSPQQPPQTRWVAPRNRNAAFGQSGGAGSDSNSPGNVQPNSAPSVESHPVLEKLKAAHSYNPKEFEWNLKSGRVFIIKSYSEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 2 - protein, 201 aa, Artificial Sequence
  The protein YTHDFl (361-559)
GPSVESHPVLEKLKAAHSYNPKEFEWNLKSGRVFIIKSYSEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 3 - protein, 238 aa, Artificial Sequence
  HisYTHDFl (361-559)
HHHHHHSSGLVPRGSHMASMTGGQQMGRGSEFENLYFQGSVESHPVLEKLKAAHSYNPKEFEWNLKSGRVFIIKSYSEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 4 - protein, 201 aa, Artificial Sequence
  YTHDF1 Mutant
GPSVESHPVLEKLKAAHSYNPKEFEANLKSGRVFIIKSYSEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 5 - protein, 201 aa, Artificial Sequence
  YTHDF1 Mutant
GPSVESHPVLEKLKAAHSYNPKEFEWNLKSGRVFIIKSYSEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKATTSIFDDFAHYEKRQEEEEVVRKERQSRNKQ

SEQ ID NO: 6 - protein, 201 aa, Artificial Sequence
  YYTHDF1 Mutant
GPSVESHPVLEKLKAAHSYAPKEFEWNLKSGRVFIIKSYSEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 7 - protein, 201 aa, Artificial Sequence
  YTHDF1 Mutant
GPSVESHPVLEKLKAAHSYNPKEFEWNLKSGRVFIIKSYSEDDIHRSIKYSIWCSTEHGNKRLDSAFRCMSSKGPVYLLFSVNGSGHFCGVAEMKSPVDYGTSAGVWSQDKWKGKFDVQWIAVKDVPNNQLRHIRLENNDNKPVTNSRDTQEVPLEKAKQVLKIISSYKHTTSIFDDFAHYEKRQEEEEVVRKERQSRNKQ

SEQ ID NO: 8 - protein, 201 aa, Artificial Sequence
  YTHDF1 Mutant
GPSVESHPVLEKLKAAHSYNPKEAEWNLKSGRVFIIKSYSEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 9 - protein, 201 aa, Artificial Sequence
  YTHDF1 Mutant
GPSVESHPVLEKLKAAHSYNPKEFEWNLKSGRVFIIASYSEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 10 - protein, 201 aa, Artificial Sequence
  YTHDF1 Mutant
GPSVESHPVLEKLKAAHSYNPKEFEWNLKSGRVFIIKSASEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 11 - protein, 201 aa, Artificial Sequence
  YTHDF1 Mutant
GPSVESHPVLEKLKAAHSYNPKEFEWNLKSGRVFIIKSYSEDDIHRSIKYSIWASTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 12 - protein, 201 aa, Artificial Sequence
  YTHDF1 Mutant
GPSVESHPVLEKLKAAHSYNPKEFEWNLKSGRVFIIKSYSEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSADTQEVPLEKAKQVLKIISSYKHTTSIFDDFAHYEKRQEEEEVVRKERQSRNKQ

SEQ ID NO: 13 - protein, 174 aa, Artificial Sequence
  YTHDF1 Mutant
GPSVESHPVLEKLKAAHSYNPKEFEWNLKSGRVFIIKSYSEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSI

SEQ ID NO: 14 - RNA, 17 nt, Artificial Sequence
  mRNA
  modified_base (1)..(1): FAM labeled u
  modified_base (12)..(12): m6a
uucuucuguggacugug

SEQ ID NO: 15 - RNA, 17 nt, Artificial Sequence
  mRNA
  modified_base (1)..(1): biotinylated u
  modified_base (12)..(12): m6a
uucuucuguggacugug

SEQ ID NO: 16 - protein, 21 aa, Artificial Sequence
  Y1 fragment or derivative of YTHDF1
KAAHSYNPKEFEWNLKSGRVF

SEQ ID NO: 17 - protein, 16 aa, Artificial Sequence
  Y2 fragment or derivative of YTHDF1
IFVKDVPNNQLRHIRL

SEQ ID NO: 18 - protein, 10 aa, Artificial Sequence
  Y3 fragment or derivative of YTHDF1
YKHTTSIFDD

(19) United States
(12) Patent Application Publication
(10) Pub. No.: US 2024/0033244 A1
(43) Pub. Date: Feb. 1, 2024

(54) COMPOSITIONS AND METHODS FOR INHIBITING YTHDF1

(71) Applicants: SHANGHAI INSTITUTE OF MATERIA MEDICA, CHINESE ACADEMY OF SCIENCES, Shanghai (CN); HANGZHOU LEADING EDGE PHARMACEUTICAL LTD., Hangzhou (CN); SHANGHAI KANGQIAN BIOTECHNOLOGY LIMITED, Shanghai (CN)

(72) Inventors: Cheng LUO, Shanghai (CN); Meng XU, Hangzhou (CN); Shijie CHEN, Shanghai (CN); Yilin LI, Hangzhou (CN); Yantao CHEN, Shanghai (CN); Hualiang JIANG, Shanghai (CN); Kaixian CHEN, Shanghai (CN); Zhanpeng JIANG, Shanghai (CN)

(73) Assignees: SHANGHAI INSTITUTE OF MATERIA MEDICA, CHINESE ACADEMY OF SCIENCES, Shanghai (CN); HANGZHOU LEADING EDGE PHARMACEUTICAL LTD., Hangzhou (CN); SHANGHAI KANGQIAN BIOTECHNOLOGY LIMITED, Shanghai (CN)

(21) Appl. No.: 18/004,623

(22) PCT Filed: Jul. 8, 2021
(86) PCT No.: PCT/CN2021/105208

§ 371 Date: Jan. 6, 2023

(30) Foreign Application Priority Data
Jul. 9, 2020 (WO) PCT/CN2020/101106

(51) Int. Cl. A61K 31/343; A61K 35/15; A61K 39/395; A61K 31/216; A61P 35/00

(52) U.S. Cl. CPC A61K 31/343; A61K 35/15; A61K 39/3955; A61K 31/216; A61P 35/00

(57) ABSTRACT

A YTH N6-Methyladenosine RNA Binding Protein 1 (YTHDF1) attenuating agent, with a compound, and when bound to YTHDF1, the compound binds to amino acid residues 372-392, 479-494 and 526-535 of SEQ ID NO: 1. A modified antigen presenting cell (mAPC), with the mAPC being treated with a YTHDF1 attenuating agent. A composition, with a YTHDF1 attenuating agent, a mAPC treated with the YTHDF1 attenuating agent, and optionally a pharmaceutically acceptable carrier. A method for attenuating an activity of YTHDF1, by administering an effective amount of a YTHDF1 attenuating agent. A method for determining whether or not a candidate agent is a YTHDF1 attenuating agent, by contacting the candidate agent with a YTHDF1 mutant. A method for treating a disease, disorder or condition associated with an expression of an antigen in a subject in need thereof.

### BACKGROUND

Spontaneous T cell priming against tumor neoantigens is critical for the clinical efficacy of immunotherapies. However, in many patients, neoantigen recognition is insufficient to induce the lasting T cell response required for complete tumor rejection. Identifying molecular pathways that influence the immunoreactivity to tumor neoantigen could provide targets for improving the response to immunotherapy. For example, m⁶A, the most abundant internal mRNA modification, is responsible for posttranscriptional regulation of mRNA in diverse cell types. Additionally, m⁶A can affect mRNA translation efficiency via the m⁶A binding protein YTH domain-containing family protein 1 (YTHDF1). Previous studies have shown that attenuating the activity of YTHDF1 in various cells of the immune system (e.g., antigen presenting cells) might be useful for inducing a sufficient and lasting antitumor immune response. However, effective compositions and methods for attenuating the activity of YTHDF1 are still highly desired.

### SUMMARY OF THE INVENTION

The present application provides compositions and methods for attenuating the activity of YTHDF1. The present application also provides modified antigen presenting cell (mAPC), such as modified dendritic cells, with enhanced activity. The compositions and mAPCs of the present application may be used for one or more of the following: activating an APC (such as a DC); generating an immune cell having enhanced anti-tumor activity; preventing and/or reversing exhaustion of an immune cell (such as T cell); treating a disease, disorder or condition associated with an expression of an antigen in a subject in need thereof; treating cancer in a subject in need thereof; stimulating a T cell-mediated immune response to a cancer cell and/or a tumor antigen in a subject in need thereof; providing an anti-tumor immunity in a subject in need thereof; increasing and/or improving proliferation and/or activity of tumor infiltrating T cells; increasing and/or improving proliferation and/or activity of tumor specific T cell; enhancing cytokine production of T cells; 12) enhancing the antitumor response of a cancer immunotherapy; and 13) inhibiting tumor growth, inhibiting the proliferation of tumor cells, and/or killing tumor cells. The present application also provides methods and compositions for enhancing an immune response with a combination of the YTHDF1 attenuating agent of the present application and a second active agent, such as an immune checkpoint inhibitor.

YTHDF1, a member of the YTH domain family, is a “reader” of m⁶A modification. By e.g., interacting with translation initiation factors, YTHDF1 helps to promote the translation efficiency of mRNA. Further, dysregulation of YTHDF1 might break the expression balance between oncogenes and tumor suppressors, implying the link between YTHDF1 and tumorigenesis. It has been reported that overexpression of YTHDF1 is associated with some malignant tumors like colorectal cancer (CRC) and hepatocellular carcinoma (HCC). Moreover, it was found that Ythdf1-deficient (Ythdf1^{-/-}) mice exhibited an elevated anti-tumor immunity response, implicating that YTHDF1 is a new potential therapeutic target. YTHDF1 has also been found to be associated with the expression of T cell exhaustion signature genes. Mice lacking YTHDF1 in T cells demonstrated better anti-tumor immunity for lymphoma, solid tumors (such as melanoma and colon cancer) and other types of cancers. Functions of tumor-infiltrating T cell were enhanced in YTHDF1-deficient mice. Furthermore, the divergence of T cell exhaustion was rescued toward a fate of memory-like or stem-like CD8⁺ T cell.

In one aspect, the present application provides a YTH N6-Methyladenosine RNA Binding Protein 1 (YTHDF1) attenuating agent, the agent comprises a compound, and when bound to YTHDF1, the compound binds to at least one residue corresponding to a residue selected from amino acid residues 372-392, 479-494 and 526-535 of SEQ ID NO: 1.

In some embodiments, when bound to YTHDF1, the compound comprised by the YTHDF1 attenuating agent binds to at least one residue corresponding to the following residues: N378, F382, W384, F480, and H528 of SEQ ID NO: 1.

In some embodiments, the compound comprised by the YTHDF1 attenuating agent is capable of blocking binding of YTHDF1 to m⁶A.

In some embodiments, the compound comprised by the YTHDF1 attenuating agent does not substantially compete with m⁶A for binding to YTHDF1.

In some embodiments, the YTHDF1 attenuating agent comprises a compound of Formula I, a prodrug, a metabolite, a derivative of the compound of Formula I, or a pharmaceutically acceptable salt, ester, or amide of any of the foregoing:

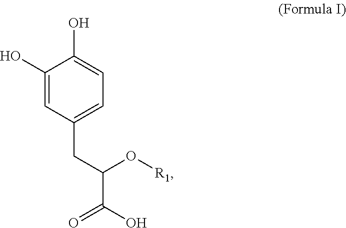

wherein R₁ is selected from the group consisting of C₁₋₅₀ hydrocarbyl, C₁₋₅₀ substituted hydrocarbyl, C₁₋₅₀ heterohydrocarbyl and C₁₋₅₀ substituted heterohydrocarbyl.

In some embodiments, R₁ in Formula I is (CO)—R₂, and R₂ is an optionally substituted alkenyl. In some embodiments, R₂ is CH═CH—R₃, and R₃ is an optionally substituted aryl. In some embodiments, R₃ is of Formula II

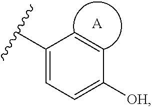

wherein A is an optionally substituted furan, or

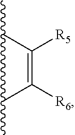

R₆ is hydroxyl, and R₅ is an optionally substituted alkenyl.

In some embodiments, in Formula II, A is

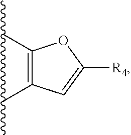

### and R₄ is

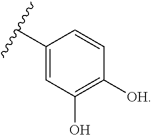 [SMILES: *c1ccc(O)c(O)c1]

In some embodiments, in Formula II, A is

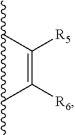

R₆ is hydroxyl, R₅ is CH═CH—R₇, and R₇ is

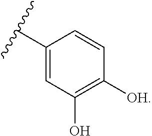 [SMILES: *c1ccc(O)c(O)c1]

In some embodiments, the compound comprised by the YTHDF1 attenuating agent of the present application comprises at least two dihydroxyphenyl moieties.

In some embodiments, the compound comprised by the YTHDF1 attenuating agent comprises at least three dihydroxyphenyl moieties.

In some embodiments, the YTHDF1 attenuating agent comprises a compound of Formula III, a prodrug, a metabolite, a derivative of the compound of Formula III, or a pharmaceutically acceptable salt, ester, or amide of any of the foregoing:

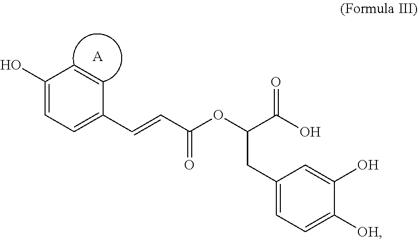

wherein A is an optionally substituted furan, or

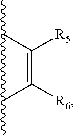

R₆ is hydroxyl, and R₅ is an optionally substituted alkenyl.

In some embodiments, in Formula III, A is

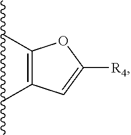

### and R₄ is

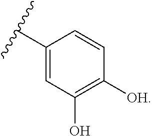 [SMILES: *c1ccc(O)c(O)c1]

In some embodiments, in Formula III, A is

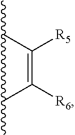

R₆ is hydroxyl, R₅ is CH═CH—R₇, and R₇ is

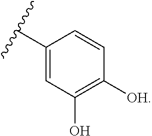 [SMILES: *c1ccc(O)c(O)c1]

In some embodiments, the compound comprises any of the following compounds, a prodrug, a metabolite, a derivative of any of the following compounds, or a pharmaceutically acceptable salt, ester, or amide of any of the foregoing:

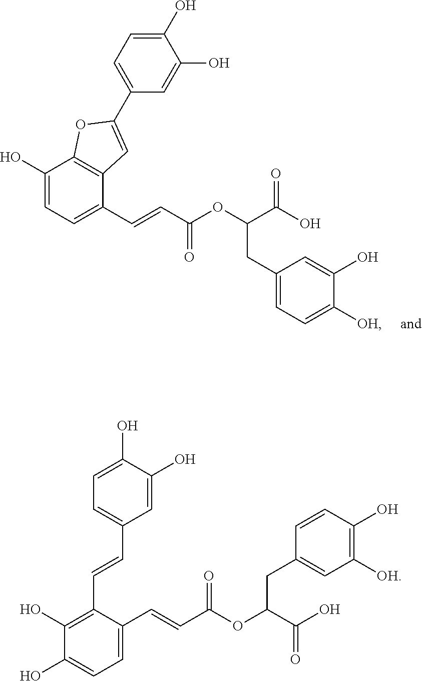 [SMILES: O=C(/C=C/c1ccc(O)c2oc(-c3ccc(O)c(O)c3)cc12)OC(Cc1ccc(O)c(O)c1)C(=O)O ; "and" ; O=C(/C=C/c1ccc(O)c(O)c1/C=C/c1ccc(O)c(O)c1)OC(Cc1ccc(O)c(O)c1)C(=O)O]

In some embodiments, the compound comprises any of the following compounds, a prodrug, a metabolite, a derivative of any of the following compounds, or a pharmaceutically acceptable salt, ester, or amide of any of the foregoing:

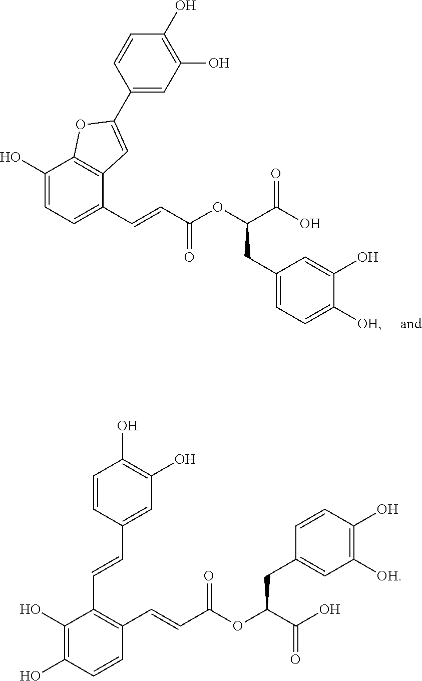 [SMILES: O=C(/C=C/c1ccc(O)c2oc(-c3ccc(O)c(O)c3)cc12)O[C@H](Cc1ccc(O)c(O)c1)C(=O)O ; "and" ; O=C(/C=C/c1ccc(O)c(O)c1/C=C/c1ccc(O)c(O)c1)O[C@@H](Cc1ccc(O)c(O)c1)C(=O)O]

In some embodiments, the compound comprises any of the following compounds, a prodrug, a metabolite, a derivative of any of the following compounds, or a pharmaceutically acceptable salt, ester, or amide of any of the foregoing:

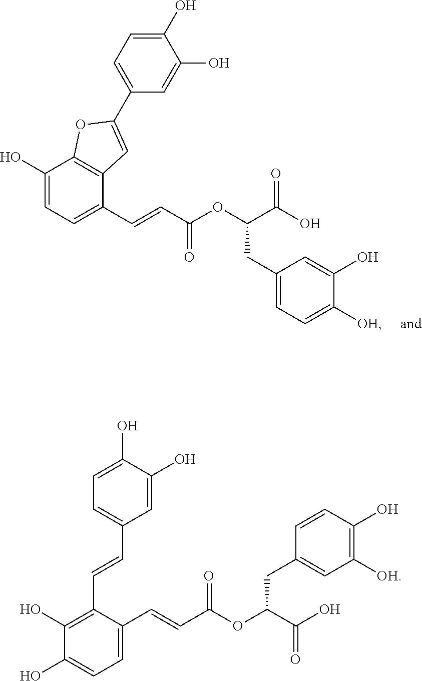 [SMILES: O=C(/C=C/c1ccc(O)c2oc(-c3ccc(O)c(O)c3)cc12)O[C@@H](Cc1ccc(O)c(O)c1)C(=O)O ; "and" ; O=C(/C=C/c1ccc(O)c(O)c1/C=C/c1ccc(O)c(O)c1)O[C@H](Cc1ccc(O)c(O)c1)C(=O)O]

In some embodiments, the compound comprised by the YTHDF1 attenuating agent is plant derived.

In some embodiments, the compound comprised by the YTHDF1 attenuating agent is provided in a plant extract. In some embodiments, the plant is of the genus Salvia. In some embodiments, the plant is Salvia miltiorrhiza (Danshen).

In one aspect, the present application provides a modified antigen presenting cell (mAPC), wherein the mAPC has been treated with and/or comprises a YTHDF1 attenuating agent of the present application. In some embodiments, the mAPC is a modified dendritic cell (mDC).

In one aspect, the present application provides a composition, comprising a YTHDF1 attenuating agent of the present application, and/or a mAPC of the present application. In some embodiments, the composition of the present application comprises a pharmaceutically acceptable carrier. In some embodiments, the composition is a vaccine composition.

In some embodiments, the composition further comprises, a second active ingredient. In some embodiments, the second active ingredient is an anti-cancer agent.

In some embodiments, the second active ingredient comprises a cancer immunotherapy. In some embodiments, the second active ingredient comprises an immune checkpoint inhibitor. In some embodiments, the second active ingredient comprises an agent selected from the group consisting of: an anti-PD-L1 antibody or an antigen binding portion thereof, an anti-PD-1 antibody or an antigen binding portion thereof, an anti-CTLA-4 antibody or an antigen binding portion thereof, and an IDO inhibitor.

In some embodiments, the second active ingredient comprises pembrolizumab, nivolumab, cemiplimab, atezolizumab, avelumab, durvalumab, ipilimumab, and/or an antigen binding fragment or a derivative of any of the foregoing.

In some embodiments, the second active ingredient is capable of causing an increase of one or more tumor antigens in a subject receiving it.

In some embodiments, the tumor antigen is selected from the group consisting of CEA, gp100, the MAGE family of proteins, DAGE, GAGE, RAGE, NY-ESO 1, Melan-A/MART 1, TRP-1, TRP-2, tyrosinase, HER-2/neu, MUC-1, p53, KSA, PSA, PSMA, and fragments and modified versions thereof.

In some embodiments, the second active ingredient is comprised in a separate container and is not mixed with the mAPC, or with the YTHDF1 attenuating agent.

In one aspect, the present application provides a method for attenuating an activity of YTHDF1, comprising administering an effective amount of a YTHDF1 attenuating agent of the present application.

In some embodiments, the method is an in vivo method. In some embodiments, the method is an in vitro method. In some embodiments, the method is an ex vivo method.

In one aspect, the present application provides a method for determining whether or not a candidate agent is a YTHDF1 attenuating agent, comprising: contacting the candidate agent with a YTHDF1 mutant, wherein the YTHDF1 mutant comprises one or more amino acid substitution, deletion and/or addition at one or more residues corresponding to a residue selected from residues 372-392, 479-494 and 526-535 of SEQ ID NO: 1.

In some embodiments, the YTHDF1 mutant comprises one or more amino acid substitution, deletion and/or addition at one or more residues corresponding to a residue selected from residues N378, F382, W384, F480, and H528 of SEQ ID NO: 1.

In some embodiments, the method further comprises determining whether or not the candidate agent specifically binds to the YTHDF1 mutant.

In one aspect, the present application provides a kit, comprising a YTHDF1 mutant of the present application.

In one aspect, the present application provides use of a compound in the manufacture of a YTHDF1 attenuating agent, wherein, when bound to YTHDF1, the compound binds to at least one residue corresponding to a residue selected from amino acid residues 372-392, 479-494 and 526-535 of SEQ ID NO: 1.

In some embodiments, when bound to YTHDF1, the compound binds to at least one residue corresponding to the following residues: N378, F382, W384, F480, and H528 of SEQ ID NO: 1.

In some embodiments, the compound is capable of blocking binding of YTHDF1 to m⁶A.

In some embodiments, the compound does not substantially compete with m⁶A for binding to YTHDF1.

In some embodiments, the compound comprises a compound of Formula I, a prodrug, a metabolite, a derivative of the compound of Formula I, or a pharmaceutically acceptable salt, ester, or amide of any of the foregoing:

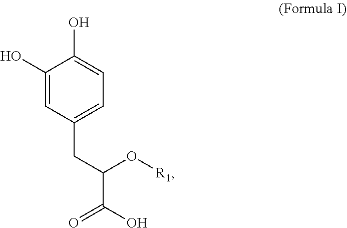

wherein R₁ is selected from the group consisting of C₁₋₅₀ hydrocarbyl, C₁₋₅₀ substituted hydrocarbyl, C₁₋₅₀ heterohydrocarbyl and C₁₋₅₀ substituted heterohydrocarbyl.

In some embodiments, in the compound of Formula I, R₁ is (CO)—R₂, and R₂ is an optionally substituted alkenyl. In some embodiments, R₂ is CH═CH—R₃, and R₃ is an optionally substituted aryl.

In some embodiments, R₃ is of Formula II

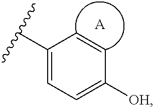

wherein A is an optionally substituted furan, or

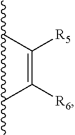

R₆ is hydroxyl, and R₅ is an optionally substituted alkenyl.

In some embodiments, in Formula II, A is

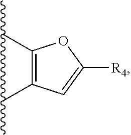

### and R₄ is

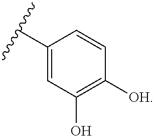 [SMILES: *c1ccc(O)c(O)c1]

In some embodiments, in Formula II, A is

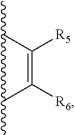

R₆ is hydroxyl, R₅ is CH═CH—R₇, and R₇ is

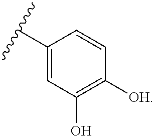 [SMILES: *c1ccc(O)c(O)c1]

In some embodiments, the compound comprises at least two dihydroxyphenyl moieties.

In some embodiments, the compound comprises at least three dihydroxyphenyl moieties.

In some embodiments, the compound comprises a compound of Formula III, a prodrug, a metabolite, a derivative of the compound of Formula III, or a pharmaceutically acceptable salt, ester, or amide of any of the foregoing:

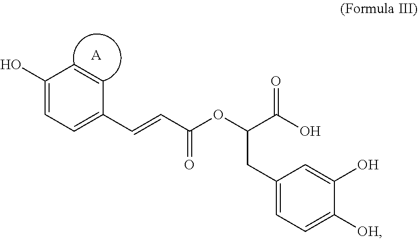

wherein A is an optionally substituted furan, or

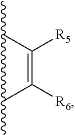

R₆ is hydroxyl, and R₅ is an optionally substituted alkenyl.

In some embodiments, in Formula III, A is

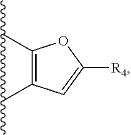

### and R₄ is

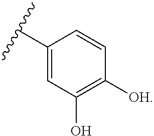 [SMILES: *c1ccc(O)c(O)c1]

In some embodiments, A is

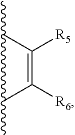

R₆ is hydroxyl, R₅ is CH═CH—R₇, and R₇ is

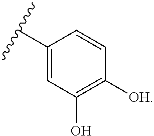 [SMILES: *c1ccc(O)c(O)c1]

In some embodiments, the compound comprises any of the following compounds, a prodrug, a metabolite, a derivative of any of the following compounds, or a pharmaceutically acceptable salt, ester, or amide of any of the foregoing:

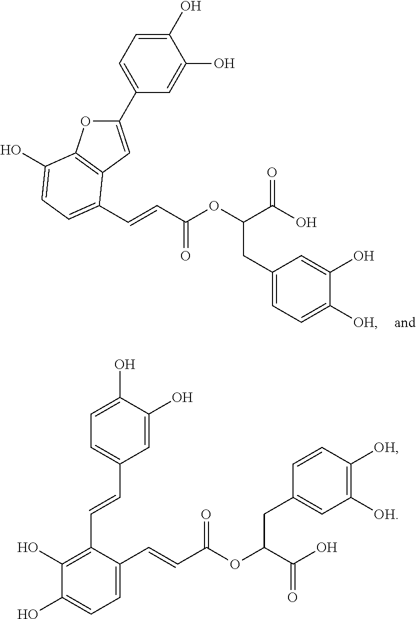 [SMILES: O=C(/C=C/c1ccc(O)c2oc(-c3ccc(O)c(O)c3)cc12)OC(Cc1ccc(O)c(O)c1)C(=O)O ; "and" ; O=C(/C=C/c1ccc(O)c(O)c1/C=C/c1ccc(O)c(O)c1)OC(Cc1ccc(O)c(O)c1)C(=O)O]

In some embodiments, the compound is plant derived. In some embodiments, the compound is provided in a plant extract. In some embodiments, the plant is of the genus Salvia. In some embodiments, the plant is Salvia miltiorrhiza (Danshen).

In one aspect, the present application provides a method for activating an APC, the method comprises administering a YTHDF1 attenuating agent of the present application to the APC.

In one aspect, the present application provides a method for activating a DC, the method comprises administering a YTHDF1 attenuating agent of the present application to the DC.

In one aspect, the present application provides a method for treating a disease, disorder or condition associated with an expression of an antigen in a subject in need thereof, comprising administering to the subject a YTHDF1 attenuating agent of the present application, a mAPC of the present application, and/or a composition of the present application.

In some embodiments of the method, the antigen is a tumor antigen.

In some embodiments of the method, the antigen is a tumor antigen selected from the group consisting of CEA, gp100, the MAGE family of proteins, DAGE, GAGE, RAGE, NY-ESO 1, Melan-A/MART 1, TRP-1, TRP-2, tyrosinase, HER-2/neu, MUC-1, p53, KSA, PSA, PSMA, and fragments and modified versions thereof.

In some embodiments of the method, the disease, disorder or condition is cancer.

In some embodiments, the cancer is selected from the group consisting of a hematological cancer, a lymphoma, and a solid tumor.

In some embodiments, the cancer is selected from the group consisting of melanoma, breast cancer, lung cancer, ovarian cancer, brain cancer, liver cancer, cervical cancer, colon cancer, colorectal cancer, renal cancer, skin cancer, head & neck cancer, bone cancer, esophageal cancer, bladder cancer, uterine cancer, lymphatic cancer, stomach cancer, pancreatic cancer, testicular cancer, lymphoma, and leukemia.

In one aspect, the present application provides a method for treating cancer in a subject in need thereof, comprising administering to the subject: a YTHDF1 attenuating agent of the present application, a mAPC of the present application, and/or a composition of the present application. In some embodiments, the cancer is selected from the group consisting of a hematological cancer, a lymphoma, and a solid tumor. In some embodiments, the cancer is selected from the group consisting of melanoma, breast cancer, lung cancer, ovarian cancer, brain cancer, liver cancer, cervical cancer, colon cancer, colorectal cancer, renal cancer, skin cancer, head & neck cancer, bone cancer, esophageal cancer, bladder cancer, uterine cancer, lymphatic cancer, stomach cancer, pancreatic cancer, testicular cancer, lymphoma, and leukemia.

In one aspect, the present application provides a method for stimulating a T cell-mediated immune response to a cancer cell and/or a tumor antigen in a subject in need thereof, comprising administering to the subject a YTHDF1 attenuating agent of the present application, a mAPC of the present application, and/or a composition of the present application. In some embodiments, the tumor antigen is selected from the group consisting of: CEA, gp100, the MAGE family of proteins, DAGE, GAGE, RAGE, NY-ESO 1, Melan-A/MART 1, TRP-1, TRP-2, tyrosinase, HER-2/neu, MUC-1, p53, KSA, PSA, PSMA, and fragments and modified versions thereof.

In one aspect, the present application provides a method for providing an anti-tumor immunity in a subject in need thereof, comprising administering to the subject a YTHDF1 attenuating agent of the present application, a mAPC of the present application, and/or a composition of the present application.

In one aspect, the present application provides a method for preventing and/or reversing exhaustion of T cells in a subject in need thereof, comprising administering to the subject a YTHDF1 attenuating agent of the present application, a mAPC of the present application, and/or a composition of the present application.

In one aspect, the present application provides a method for enhancing an activity of T cells in a subject in need thereof, comprising administering to the subject a YTHDF1 attenuating agent of the present application, a mAPC of the present application, and/or a composition of the present application. In some embodiments, the T cells comprises tumor infiltrating T cells. In some embodiments, the T cells comprises tumor specific T cells.

In some embodiments of the method of the present application, the subject is a cancer patient. In some embodiments, the cancer is selected from the group consisting of a hematological cancer, a lymphoma, and a solid tumor. In some embodiments, the cancer is selected from the group consisting of melanoma, breast cancer, lung cancer, ovarian cancer, brain cancer, liver cancer, cervical cancer, colon cancer, colorectal cancer, renal cancer, skin cancer, head & neck cancer, bone cancer, esophageal cancer, bladder cancer, uterine cancer, lymphatic cancer, stomach cancer, pancreatic cancer, testicular cancer, lymphoma, and leukemia.

In some embodiments, the subject has received, is receiving, and/or will receive an anti-cancer treatment. In some embodiments, the anti-cancer treatment comprises a cancer immunotherapy. In some embodiments, the anti-cancer treatment comprises an immune checkpoint inhibitor. In some embodiments, the anti-cancer treatment comprises an agent selected from the group consisting of: an anti-PD-L1 antibody or an antigen binding portion thereof, an anti-PD-1 antibody or an antigen binding portion thereof, an anti-CTLA-4 antibody or an antigen binding portion thereof, and an IDO inhibitor. In some embodiments, the anti-cancer treatment comprises pembrolizumab, nivolumab, cemiplimab, atezolizumab, avelumab, durvalumab, ipilimumab, and/or an antigen binding fragment or a derivative of any of the foregoing. In some embodiments, the anti-cancer treatment is capable of causing an increase of one or more tumor antigens in the subject. In some embodiments, the tumor antigen is selected from the group consisting of: CEA, gp100, the MAGE family of proteins, DAGE, GAGE, RAGE, NY-ESO 1, Melan-A/MART 1, TRP-1, TRP-2, tyrosinase, HER-2/neu, MUC-1, p53, KSA, PSA, PSMA, and fragments and modified versions thereof.

In some embodiments, the method further comprises administering to the subject one or more additional anti-cancer treatment. In some embodiments, the additional anti-cancer treatment comprises a cancer immunotherapy. In some embodiments, the additional anti-cancer treatment comprises an immune checkpoint inhibitor. In some embodiments, the additional anti-cancer treatment comprises an agent selected from the group consisting of: an anti-PD-L1 antibody or an antigen binding portion thereof, an anti-PD-1 antibody or an antigen binding portion thereof, an anti-CTLA-4 antibody or an antigen binding portion thereof, and an IDO inhibitor. In some embodiments, the additional anti-cancer treatment comprises pembrolizumab, nivolumab, cemiplimab, atezolizumab, avelumab, durvalumab, ipilimumab, and/or an antigen binding fragment or a derivative of any of the foregoing. In some embodiments, the additional anti-cancer treatment is capable of causing an increase of one or more tumor antigens in the subject. In some embodiments, the tumor antigen is selected from the group consisting of: CEA, gp100, the MAGE family of proteins, DAGE, GAGE, RAGE, NY-ESO 1, Melan-A/MART 1, TRP-1, TRP-2, tyrosinase, HER-2/neu, MUC-1, p53, KSA, PSA, PSMA, and fragments and modified versions thereof.

In one aspect, the present application provides use of a YTHDF1 attenuating agent of the present application, a mAPC of the present application, and/or a composition of the present application in the manufacture of a composition and/or of a medicament for one or more of the following: 1) activating an APC; 2) activating a DC; 3) generating an immune cell having enhanced anti-tumor activity; 4) preventing and/or reversing exhaustion of an immune cell (such as T cell); 5) treating a disease, disorder or condition associated with an expression of an antigen in a subject in need thereof; 6) treating cancer in a subject in need thereof; 7) stimulating a T cell-mediated immune response to a cancer cell and/or a tumor antigen in a subject in need thereof; 8) providing an anti-tumor immunity in a subject in need thereof; 9) increasing and/or improving proliferation and/or activity of tumor infiltrating T cells; 10) increasing and/or improving proliferation and/or activity of tumor specific T cell; 11) enhancing cytokine production of T cells; 12) enhancing the antitumor response of a cancer immunotherapy; and 13) inhibiting tumor growth, inhibiting the proliferation of tumor cells, and/or killing tumor cells.

In some embodiments, the cancer or tumor is selected from the group consisting of a hematological cancer, a lymphoma, and a solid tumor. In some embodiments, the cancer or tumor is selected from the group consisting of melanoma, breast cancer, lung cancer, ovarian cancer, brain cancer, liver cancer, cervical cancer, colon cancer, colorectal cancer, renal cancer, skin cancer, head & neck cancer, bone cancer, esophageal cancer, bladder cancer, uterine cancer, lymphatic cancer, stomach cancer, pancreatic cancer, testicular cancer, lymphoma, and leukemia.

In one aspect, the present application provides use of a YTHDF1 attenuating agent of the present application, a mAPC of the present application, and/or a composition of the present application in combination with an additional active ingredient in the manufacture of a medicament for one or more of the following: 1) activating an APC; 2) activating a DC; 3) generating an immune cell having enhanced anti-tumor activity; 4) preventing and/or reversing exhaustion of an immune cell (such as T cell); 5) treating a disease, disorder or condition associated with an expression of an antigen in a subject in need thereof; 6) treating cancer in a subject in need thereof; 7) stimulating a T cell-mediated immune response to a cancer cell and/or a tumor antigen in a subject in need thereof; 8) providing an anti-tumor immunity in a subject in need thereof; 9) increasing and/or improving proliferation and/or activity of tumor infiltrating T cells; 10) increasing and/or improving proliferation and/or activity of tumor specific T cell; 11) enhancing cytokine production of T cells; 12) enhancing the antitumor response of a cancer immunotherapy; and 13) inhibiting tumor growth, inhibiting the proliferation of tumor cells, and/or killing tumor cells.

In some embodiments, the additional active ingredient comprises a cancer immunotherapy. In some embodiments, the additional active ingredient comprises an immune checkpoint inhibitor. In some embodiments, the additional active ingredient comprises an agent selected from the group consisting of: an anti-PD-L1 antibody or an antigen binding portion thereof, an anti-PD-1 antibody or an antigen binding portion thereof, an anti-CTLA-4 antibody or an antigen binding portion thereof, and an IDO inhibitor. In some embodiments, the additional active ingredient comprises pembrolizumab, nivolumab, cemiplimab, atezolizumab, avelumab, durvalumab, ipilimumab, and/or an antigen binding fragment or a derivative of any of the foregoing. In some embodiments, the additional active ingredient is capable of causing an increase of one or more tumor antigens in a subject receiving it. In some embodiments, the tumor antigen is selected from the group consisting of: CEA, gp100, the MAGE family of proteins, DAGE, GAGE, RAGE, NY-ESO 1, Melan-A/MART 1, TRP-1, TRP-2, tyrosinase, HER-2/neu, MUC-1, p53, KSA, PSA, PSMA, and fragments and modified versions thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are employed, and the accompanying drawings (also “figure” and “FIG.” herein), of which:

FIGS. 1a-1b illustrate the inhibitory activity of SAA. FIG. 1c illustrates the inhibitory activity of SAC. FIG. 1d illustrates the binding of SAA to YTHDF1.

FIGS. 2a-2c illustrate the ITC binding curve of SAA binding to YTHDF1.

FIG. 3a illustrates the results of SPR binding assay between SAA and YTHDF1. FIGS. 3b-3c illustrate the results of MST binding assay between SAA and YTHDF1.

FIGS. 4a-4b illustrate the results of competitive binding analysis between SAA and m⁶A-containing mRNA.

FIG. 5a illustrates the residue plot of HDX MS experiment results. FIG. 5b illustrates the butterfly plot of HDX MS experiment results.

FIG. 6 illustrates the heat map of HDX MS experiment results.

FIG. 7 illustrates the local deuterium uptake kinetics of YTHDF1. On the right, the vertical axis represents the percentage of deuterium uptake and the horizontal axis indicates the duration of HDX process. A paired t test was used to compare the variation of deuterium uptake, and p<0.05 (*) was considered to be statistically significant.

FIGS. 8a-8i illustrate the local exchange kinetics analysis of relative peptides. The vertical axis represents the percentage of deuterium uptake and the horizontal axis indicates the duration of HDX process.

FIG. 9 illustrates the inhibitory activities of SAA against YTHDF1 mutants and truncation. The IC₅₀ values of SAA were determined by FP assay. Data were expressed as the mean±s.e.m.

FIGS. 10a-10j illustrate the IC₅₀ values of YTHDF1 mutants and C-terminal truncation measured by FP assay. SAA was diluted from 100 μM via a two-fold gradient dilution.

FIGS. 11a-11g illustrate the K_{d} values of YTHDF1 and its mutants or C-terminal truncation measured via FP assay. The protein was diluted from 200 μM by a two-fold gradient dilution.

FIGS. 12a-12b illustrate binding of SAA to YTHDF1 in 293T cells. CETSA assay was performed in 293T cell line, the temperature ranged from 39.0° C. to 59.0° C. as indicated. The quantity of YTHDF1 was detected via western blot with GAPDH as the internal reference. And the relative quantification according to the western blot (FIG. 12a) is shown in FIG. 12b.

FIGS. 13a-13b illustrate the anti-tumor effects of SAA, which depend on T cells and DCs.

FIGS. 14a-14b illustrate the effects of SAA on tumor cell growth in vitro.

FIG. 15a illustrates the effects of SAA in Rag1^{-/-} mice. FIGS. 15b-15c illustrate the ability of SAA to enhance cross-priming of T cells by DCs.

FIGS. 16a-16b illustrate the ability of SAA to enhance direct-priming of T cells by DCs.

FIG. 17a illustrates that SAA targets DCs to inhibit tumor growth. FIGS. 17b-17c illustrate that SAA could enhance the activity of tumor infiltrating T cells.

FIG. 18a illustrates that PD-1^{low} population from SAA treated group expressed much more CXCR5 than DMSO group. FIG. 18b illustrates that the percentage of tumor infiltrating terminally exhausted T cell (PD-1⁺Tim-3⁺) decreased in SAA treated mice. FIG. 18c illustrates the anti-tumor effects of SAA in combination with an anti-PD-L1 antibody.

FIG. 19 illustrates anti-tumor effect of SAA and SAC.

FIG. 20 illustrates anti-tumor effect of SAA together with PD-1 blockade.

FIG. 21 illustrates adoptive transfer SAA-treated FLT3L DC exhibit durable anti-tumor function.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

In one aspect, the present application provides a YTH N6-Methyladenosine RNA Binding Protein 1 (YTHDF1) attenuating agent. The YTHDF1 attenuating agent may comprise a compound. When bound to YTHDF1, the compound may bind to at least one residue corresponding to a residue selected from amino acid residues 372-392, 479-494 and 526-535 of SEQ ID NO: 1.

In one aspect, the present application provides a modified antigen presenting cell (mAPC). The mAPC may have been treated with a YTHDF1 attenuating agent of the present application. In some cases, the mAPC may comprise a YTHDF1 attenuating agent of the present application. The mAPC may be a modified dendritic cell (mDC).

In one aspect, the present application provides a composition (such as a pharmaceutical composition). The composition may comprise a YTHDF1 attenuating agent of the present application. Alternatively, or in addition, the composition may comprise a mAPC of the present application. The composition may comprise a pharmaceutically acceptable carrier. In some cases, the composition may be a vaccine composition.

In some cases, the composition may comprise an additional or a second active ingredient. In the present application, the term “additional active ingredient” and “second active ingredient” may be used interchangeably.

The additional or second active ingredient may be comprised in a separate container and is not mixed with the mAPC, or with the YTHDF1 attenuating agent of the present application.

In some cases, the additional active ingredient may be comprised in the same package or the same container as the mAPC and/or the YTHDF1 attenuating agent of the present application. In some cases, the additional active ingredient may be contained in a separate container, for example, the additional active ingredient may be contained in a container different from that containing the mAPC and/or the YTHDF1 attenuating agent of the present application. In some cases, the additional active ingredient is not in direct contact with (e.g., does not mix with) the mAPC and/or the YTHDF1 attenuating agent of the present application, even though they may be present in the same container, or in the same package.

In one aspect, the present application provides a method for attenuating an activity of YTHDF1. The method may comprise administering an effective amount of a YTHDF1 attenuating agent of the present application.

In one aspect, the present application provides a method for determining whether or not a candidate agent is a YTHDF1 attenuating agent. The method may comprise contacting the candidate agent with a YTHDF1 mutant. The YTHDF1 mutant may comprise one or more amino acid substitution, deletion and/or addition at one or more residues corresponding to a residue selected from residues 372-392, 479-494 and 526-535 of SEQ ID NO: 1.

In one aspect, the present application provides a kit. The kit may comprise a YTHDF1 mutant of the present application.

In one aspect, the present application provides use of a compound in the manufacture of a YTHDF1 attenuating agent. When bound to YTHDF1, the compound may bind to at least one residue corresponding to a residue selected from amino acid residues 372-392, 479-494 and 526-535 of SEQ ID NO: 1.

In one aspect, the present application provides a method for activating an APC. The method may comprise administering a YTHDF1 attenuating agent of the present application to the APC.

In one aspect, the present application provides a method for activating a DC. The method may comprise administering a YTHDF1 attenuating agent of the present application to the DC.

In one aspect, the present application provides a method for treating a disease, disorder or condition associated with an expression of an antigen in a subject in need thereof. The method may comprise administering to the subject a YTHDF1 attenuating agent of the present application, a mAPC of the present application, and/or a composition of the present application.

In one aspect, the present application provides a method for inhibiting tumor growth, inhibiting the proliferation of tumor cells, and/or killing tumor cells. The method may comprise administering to the tumor and/or tumor cell a YTHDF1 attenuating agent of the present application, a mAPC of the present application, and/or a composition of the present application.

In one aspect, the present application provides a method for treating cancer in a subject in need thereof. The method may comprise administering to the subject a YTHDF1 attenuating agent of the present application, a mAPC of the present application, and/or a composition of the present application.

In one aspect, the present application provides a method for stimulating a T cell-mediated immune response to a cancer cell and/or a tumor antigen (e.g., in a subject in need thereof). The method may comprise administering to the subject a YTHDF1 attenuating agent of the present application, a mAPC of the present application, and/or a composition of the present application.

In one aspect, the present application provides a method for providing an anti-tumor immunity in a subject in need thereof. The method may comprise administering to the subject a YTHDF1 attenuating agent of the present application, a mAPC of the present application, and/or a composition of the present application.

In one aspect, the present application provides a method for preventing and/or reversing exhaustion of an immune cell, such as immune effector cells (e.g., T cells) in a subject in need thereof. The method may comprise administering to the subject a YTHDF1 attenuating agent of the present application, a mAPC of the present application, and/or a composition of the present application.

In one aspect, the present application provides a method for enhancing an activity of an immune cell, such as an immune effector cell (e.g., a T cell) in a subject in need thereof. The method may comprise administering to the subject a YTHDF1 attenuating agent of the present application, a mAPC of the present application, and/or a composition of the present application. In some embodiments, the immune cells comprise tumor infiltrating T cells. In some embodiments, the immune cells comprise tumor specific T cells.

In one aspect, the present application provides use of a YTHDF1 attenuating agent of the present application, a mAPC of the present application, and/or a composition of the present application in the manufacture of a composition and/or of a medicament for one or more of the following: 1) activating an APC; 2) activating a DC; 3) generating an immune cell having enhanced anti-tumor activity; 4) preventing and/or reversing exhaustion of an immune cell (such as immune effector cells, e.g., T cells); 5) treating a disease, disorder or condition associated with an expression of an antigen in a subject in need thereof; 6) treating cancer in a subject in need thereof; 7) stimulating an immune cell (e.g., immune effector cell, such as T cell) mediated immune response to a cancer cell and/or a tumor antigen in a subject in need thereof; 8) providing an anti-tumor immunity in a subject in need thereof; 9) increasing and/or improving proliferation and/or activity of immune cells (e.g., immune effector cells, such as T cells, for example, tumor infiltrating T cells); 10) increasing and/or improving proliferation and/or activity of tumor specific immune cells (e.g., immune effector cells, such as T cells); 11) enhancing cytokine production of T cells; 12) enhancing the antitumor response of a cancer immunotherapy; and 13) inhibiting tumor growth, inhibiting the proliferation of tumor cells, and/or killing tumor cells.

In one aspect, the present application provides use of a YTHDF1 attenuating agent of the present application, a mAPC of the present application, and/or a composition of the present application in combination with an additional active ingredient in the manufacture of a medicament for one or more of the following: 1) activating an APC; 2) activating a DC; 3) generating an immune cell having enhanced anti-tumor activity; 4) preventing and/or reversing exhaustion of an immune cell (such as immune effector cells, e.g., T cells); 5) treating a disease, disorder or condition associated with an expression of an antigen in a subject in need thereof; 6) treating cancer in a subject in need thereof; 7) stimulating an immune cell (e.g., immune effector cell, such as T cell) mediated immune response to a cancer cell and/or a tumor antigen in a subject in need thereof; 8) providing an anti-tumor immunity in a subject in need thereof; 9) increasing and/or improving proliferation and/or activity of immune cells (e.g., immune effector cells, such as T cells, for example, tumor infiltrating T cells); 10) increasing and/or improving proliferation and/or activity of tumor specific immune cells (e.g., immune effector cells, such as T cells); 11) enhancing cytokine production of T cells; 12) enhancing the antitumor response of a cancer immunotherapy; and 13) inhibiting tumor growth, inhibiting the proliferation of tumor cells, and/or killing tumor cells.

The terms “comprise(s),” “include(s),” “having,” “has,” “can,” “contain(s),” and variants thereof, as used herein, generally are intended to be open-ended transitional phrases, terms, or words that do not preclude the possibility of additional acts or structures. The singular forms “a,” “and” and “the” include plural references.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4. 6.5, 6.6, 6.7, 6.8, 6.9, and 7.0 are explicitly contemplated. Accordingly, the description in range format is merely for convenience and brevity and should not be construed as an inflexible limitation on the scope of the inventions of the present application. The description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” when referring to a measurable value such as an amount, a temporal duration, and the like, is meant to encompass variations of 20% or in some instances ±10%, or in some instances ±5%, or in some instances+1%, or in some instances ±0.10% from the specified value, as such variations are appropriate.

The term “subject”, as used herein, generally refers to a human being or an animal. For example, it may refer to any vertebrate, including, but not limited to, a mammal (e.g., cow, pig, camel llama, horse, goat, rabbit, sheep. hamsters, guinea pig, cat, dog. rat, and mouse, a non-human primate (for example, a monkey, such as a cynomolgus monkey, chimpanzee, etc.) and a human). In some aspects, the subject is a human being.

The term “treat”, “treated” and “treating” may be used interchangeably herein, and generally refer to a therapeutic method wherein the object is to slow down (lessen) an undesired physiological condition, disorder or disease, or to obtain beneficial or desired clinical results. In some aspects of the present disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment also includes prolonging survival as compared to expected survival if not receiving treatment.

The terms “modified”, “modify” and “modification” may be used interchangeably herein, and generally refer to introducing or resulting in a change or alteration. When used in the context of a cell, such modification may include any conventional method for producing an alteration in the activity and/or function of the cell. For example, by exposing the cell (e.g., an antigen presenting cell) to an agent capable of adjusting the activity and/or function of the cell.

The terms “attenuating”, “attenuation” and “attenuated” may be used interchangeably, and as used herein, may refer to inhibiting or reducing the amount of or inhibiting or decreasing the activity of a target gene or a target protein (such as YTHDF1, or a target of YTHDF1). Such attenuation may be accomplished using, e.g. an antibody or a derivative thereof, an antibody-drug conjugate, a fusion protein, a small molecule, an antisense molecule, a dsRNA, a siRNA, a shRNA, an aptamer, and/or a gRNA (e.g., in combination with a gene editing system, such as with CRIPSR/Cas9). As another example, YTHDF1 may be attenuated by contacting the antigen presenting cell (e.g., a dendritic cell) with an inhibitor of YTHDF1 (such as a compound of the present application), to inhibit/block binding and/or recognizing of the m⁶A modified mRNA by YTHDF1.

The term “small molecule”, as used herein, generally refers to any chemical or other moiety, other than polypeptides and nucleic acids, that can act to affect biological processes, particularly to modulate the m⁶A mRNA modification (e.g., activity of YTHDF1). Small molecules can include any number of therapeutic agents presently known and used, or that can be synthesized in a library of such molecules for the purpose of screening for biological function(s). Small molecules are distinguished from macromolecules by size. The small molecules may have a molecular weight less than about 5,000 daltons (Da), such as less than about 2,500 Da, less than about 1,000 Da, or less than about 500 Da.

Small molecules may include without limitation organic compounds, peptidomimetics and conjugates thereof.

The term “amino acid”, as used herein in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is a 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or more posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide.

The term “YTHDF1”, as used herein, generally refers to YTH N6-Methyladenosine RNA Binding Protein 1 or a functional fragment thereof specifically recognizing and binding N6-methyladenosine (m6A)-containing RNAs, and regulating mRNA stability. The human and murine amino acid and nucleic acid sequences can be found in a public database, such as GenBank, UniProt and Swiss-Prot. For example, the amino acid sequences of human YTHDF1 can be found at Accession No. NP_060268.2, and the mRNA sequences encoding them can be found at Accession No. NM_017798.4.

The term “YTHDF1 mutant”, as used herein, generally refers to a nucleic acid molecule encoding YTHDF1, or a YTHDF1 protein, having one or more mutations therein comparing to a corresponding parent or reference (e.g., wildtype) YTHDF1 encoding nucleic acid molecule or corresponding parent or reference (e.g., wildtype) YTHDF1 protein.

In terms of a YTHDF1 mutant protein, the mutant protein has at least one amino acid residue differing from the amino acid sequence of a parent or reference polypeptide (including, but not limited to, a wild-type YTHDF1 polypeptide). A mutation in a mutant protein may include a deletion, a substitution and/or an addition of one or more amino acids. Mutations may range in size from a single amino acid to a large segment of a polypeptide. In some embodiments, an insertion changes the number of amino acids in a polypeptide by adding a piece of polypeptide. In some embodiments, a deletion changes the number of amino acids by removing a piece of polypeptide. In some embodiments, small deletions may remove one or a few amino acids within a polypeptide. In some embodiments, a substitution replaces one amino acid in a polypeptide with a different amino acid. A substitution may be conserved amino acid substitution, or non-conserved amino acid substitution. A “conserved amino acid substitution” refers to the replacement of amino acids normally present in the sequence with different amino acids of similar size, charge, polarity, and/or chemical property. Examples of conserved substitutions include the replacement of nonpolar (hydrophobic) residues such as isoleucine, valine and leucine with another nonpolar residue. Similarly, examples of conserved substitutions include arginine and lysine, glutamine and asparagine, and substitution of a polar (hydrophilic) residue, such as serine of glycine, with another polar residue. Further, substitution of a basic residue such as lysine, arginine or histidine with another basic residue or substitution of one acidic residue such as aspartic acid or glutamic acid with another acidic residue is a conserved substitution. Examples of “non-conserved substitution” may include substitution of nonpolar (hydrophobic) amino acid residues such as isoleucine, valine, leucine, alanine, methionine with polar (hydrophilic) residues such as cysteine, glutamine, glutamic acid or lysine, and/or substitution of polar residues with nonpolar residues.

The terms “cancer” and “tumor” are used herein interchangeably, and generally refer to a disease characterized by the uncontrolled growth of aberrant cells. Both terms encompass solid and liquid, e.g., diffuse or circulating, tumors. They include premalignant, as well as malignant cancers and tumors.

The phrase “disease, disorder or condition associated with an expression of an antigen” as described herein, generally includes, but is not limited to, a disease associated with expression of an antigen or condition associated with cells expressing an antigen, e.g., proliferative diseases such as a cancer or malignancy or a precancerous condition such as a myelodysplasia, a myelodysplastic syndrome or a preleukemia; or a noncancer related indication associated with cells which express or overexpress an antigen, such as antigens present in bacteria, viruses or cells, e.g., a non-cancer cell. Non-cancer related indications associated with expression of an antigen as described herein include, but are not limited to, e.g., autoimmune disease, inflammatory disorders and transplantation.

The phrase “disease, disorder or condition associated with an expression of a tumor antigen” as described herein, generally includes, but is not limited to, a disease associated with expression of a tumor antigen or condition associated with cells expressing a tumor antigen, e.g., proliferative diseases such as a cancer or malignancy or a precancerous condition such as a myelodysplasia, a myelodysplastic syndrome or a preleukemia; or a noncancer related indication associated with cells which express a tumor antigen. In one embodiment, a cancer associated with expression of a tumor antigen as described herein is a hematological cancer. In one embodiment, a cancer associated with expression of a tumor antigen as described herein is a solid cancer. Further diseases associated with expression of a tumor antigen as described herein include, but not limited to, e.g., atypical and/or non-classical cancers, malignancies, precancerous conditions or proliferative diseases associated with expression of a tumor antigen as described herein. Non-cancer related indications associated with expression of a tumor antigen as described herein include, but are not limited to, e.g., autoimmune disease, inflammatory disorders and transplantation. In some embodiments, the tumor antigen-expressing cells express, or at any time expressed, mRNA encoding the tumor antigen. In an embodiment, the tumor antigen-expressing cells produce the tumor antigen protein (e.g., wild-type or mutant), and the tumor antigen protein may be present at normal levels, elevated levels, or reduced levels.

The terms “activity” and “activating”, as used herein, generally refer to a specialized function of a cell. The activity of a T cell, for example, may be cytolytic activity or helper activity including the secretion of cytokines. The activity of an antigen presenting cell, for example, may be processing and/or presenting antigens for recognition by certain lymphocytes (such as T cells).

The term “immune effector cell”, as used herein, generally refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloic-derived phagocytes. “Immune effector function or immune effector response,” as used herein, generally refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “antigen presenting cell” or “APC”, as used herein, generally refers to a cell (e.g., an immune cell) or a group of cells (e.g., a group of immune cells) capable of displaying antigens on its or their surfaces. The displayed antigens may be complexed with major histocompatibility complexes (MHCs), and the antigens may be processed before being displayed. Examples of APCs include, but are not limited to, macrophages, B cells, and dendritic cells (such as Langerhans cells). Cellular immune responses may be initiated or enhanced after lymphocytes (e.g., T cells) recognizing the antigens presented by the APCs. APCs may break down large-molecular-weight antigens into 10 to 30 amino acid fragments for loading onto HLA class I and II molecules.

The term “dendritic cell” or “DC”, as used herein, generally refers to a type of antigen presenting cell. DCs may act as messengers between the innate and the adaptive immune systems. For example, DCs may be present in tissues that are in contact with the external environment, such as the skin, the inner lining of the nose, lungs, stomach and intestines. They can also be found in an immature state in the blood. Once activated, they may migrate to the lymph nodes where they interact with other immune cells, such as T cells and B cells to initiate and shape the adaptive immune response. Immature dendritic cells are also called veiled cells. DCs may be bone marrow (BM)-derived leukocytes. They can also be propagated in vitro from BM and blood using various combinations of growth factors, such as granulocyte macrophage-colony stimulating factor (GM-CSF) and Flt3 ligand. DCs may be specialized to capture and process antigens, converting proteins to peptides that are presented on major histocompatibility complex (MHC) molecules recognized by other immune cells, such as T cells. DCs may be heterogeneous, e.g. myeloid and plasmacytoid DCs; although all DCs may be capable of antigen uptake, processing and presentation to naïve T cells, the DC subtypes may have distinct markers and differ in location, migratory pathways, detailed immunological function and dependence on infections or inflammatory stimuli for their generation. During the development of an adaptive immune response, the phenotype and function of DCs may play an important role in initiating tolerance, memory, and/or polarised T-helper 1 (Th1), Th2 and Th17 differentiation.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “nucleic acid” or “polynucleotide”, as used herein, generally refers to deoxyribonucleic acids (DNA) or ribonucleic acid (RNA), or a combination of a DNA or RNA thereof, and polymers thereof in either single- or double-stranded form. The term “nucleic acid” includes a gene, cDNA or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “cancer associated antigen” and “tumor antigen” are used interchangeably herein and generally refer to a molecule (typically protein, carbohydrate or lipid) that is preferentially expressed on the surface of a cancer cell, either entirely or as a fragment (e.g., MHC/peptide), in comparison to a normal cell, and which is useful for the preferential targeting of a pharmacological agent to the cancer cell. In some embodiments, a tumor antigen is a marker expressed by both normal cells and cancer cells. In some embodiments, a cancer-associated antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a cancer-associated antigen is a cell surface molecule that is inappropriately synthesized in the cancer cell, for instance, a molecule that contains deletions, additions or mutations in comparison to the molecule expressed on a normal cell. In some embodiments, a cancer-associated antigen will be expressed exclusively on the cell surface of a cancer cell, entirely or as a fragment (e.g., MHC/peptide), and not synthesized or expressed on the surface of a normal cell.

The term “specifically binds,” as used herein, generally refers to a molecule (e.g., a small molecule, an antibody, or a ligand), which recognizes and binds with a cognate binding partner protein present in a sample, but which molecule does not substantially recognize or bind other molecules in the sample. In some embodiments, a molecule of the present disclosure may specifically bind to a target molecule with a binding affinity (K_{d}) of less than about 10⁻⁵ M (e.g., less than about 9×10⁻⁶ M, less than about 8×10⁻⁶ M, less than about 7×10⁻⁶ M, less than about 6×10⁻⁶ M, less than about 5×10⁻⁶ M, less than about 4×10⁻⁶ M, less than about 3.5×10⁻⁶ M, less than about 3×10⁻⁶ M, less than about 2.5×10⁻⁶ M, less than about 2×10⁻⁶ M, less than about 1×10⁻⁶ M, less than about 5×10⁻⁷ M, less than about 2×10⁻⁷ M, less than about 10⁻⁷ M, less than about 5×10⁻⁸ M, less than about 2×10⁻⁸ M, less than about 10⁻⁸ M, less than about 5×10⁻⁹ M, less than about 4×10⁻⁹M, less than about 3×10⁻⁹M, less than about 2×10⁻⁹ M, or less than about 10⁻⁹ M).

K_{d} may generally refer to the ratio of the dissociation rate to the association rate (k_{off}/kₒₙ), which may be determined by using any conventional method known in the art, including but are not limited to surface plasmon resonance method, microscale thermophoresis method, HPLC-MS method and flow cytometry (such as FACS) method. In certain embodiments, the K_{d} value can be appropriately determined by using flow cytometry.

The term “anti-cancer agent”, as used herein, generally refers to an agent that is capable of inhibiting and/or preventing the growth of a tumor or a cancer cell.

The term “CTLA-4”, as used herein, generally refers to the Cytotoxic T-lymphocyte-associated protein 4 derived from any vertebrate source, including mammals such as primates (e.g. humans, monkeys) and rodents (e.g., mice and rats), and functional fragments thereof. Exemplary sequence of human CTLA-4 includes Homo sapiens (human) CTLA-4 protein (NCBI Ref Seq No. AAL07473.1). Exemplary sequence of CTLA-4 includes Macaca fascicularis (monkey) CTLA-4 protein (NCBI Ref Seq No XP_005574071.1). The term “CTLA-4”, as used herein, generally is intended to encompass any form of CTLA-4, for example, 1) native unprocessed CTLA-4 molecule, “full-length” CTLA-4 chain or naturally occurring variants of CTLA-4, including, for example, splice variants or allelic variants; 2) any form of CTLA-4 that results from processing in the cell; or 3) full length, a fragment (e.g., a truncated form, an extracellular/transmembrane domain) or a modified form (e.g. a mutated form, a glycosylated/PEGylated, a His-tag/immunofluorescence fused form) of CTLA-4 subunit generated through recombinant method.

The term “anti-CTLA-4 antibody”, “anti-CTLA-4 binding domain” or “CTLA-4-binding domain” refers to an antibody or antigen-binding domain that is capable of specifically binding CTLA-4 (e.g. human or monkey CTLA-4).

The term “PD-1”, as used herein, generally refers programmed cell death protein, which belongs to the superfamily of immunoglobulin and functions as co-inhibitory receptor to negatively regulate the immune system. PD-1 is a member of the CD28/CTLA-4 family, and has two known ligands including PD-L1 and PD-L2. Representative amino acid sequence of human PD-1 is disclosed under the NCBI accession number: NP_005009.2, and the representative nucleic acid sequence encoding the human PD-1 is shown under the NCBI accession number: NM_005018.2.

The term “PD-L1”, as used herein, generally refers to programmed cell death ligand 1 (PD-L1, see, for example, Freeman et al. (2000) J. Exp. Med. 192: 1027). Representative amino acid sequence of human PD-L1 is disclosed under the NCBI accession number: NP_054862.1, and the representative nucleic acid sequence encoding the human PD-L1 is shown under the NCBI accession number: NM_014143.3. PD-L1 binds to its receptor PD-1 or B7-1, which is expressed on activated T cells, B cells and myeloid cells. The binding of PD-L1 and its receptor induces signal transduction to suppress TCR-mediated activation of cytokine production and T cell proliferation. Accordingly, PD-L1 plays a major role in suppressing immune system during particular events such as pregnancy, autoimmune diseases, tissue allografts, and is believed to allow tumor or cancer cells to circumvent the immunological checkpoint and evade the immune response.

The term “anti-PD-1 antibody”, “anti-PD-1 binding domain” or “PD-1 binding domain” as used herein, generally refers to an antibody or antigen-binding domain that is capable of specifically binding to PD-1 (e.g. human or monkey PD-1) with an affinity which is sufficient to provide for diagnostic and/or therapeutic use.

The term “anti-tumor immunity”, as used herein, generally refers to an immune response induced upon recognition of cancer antigens by immune cells.

The term “cancer immunotherapy”, as used herein, generally refers to any therapy that is designed to provoke or enhance an immune response against cancer cells in a patient. For example, cancer immunotherapy includes, but is not limited to, cancer antigen specific active immunotherapy, treatment with an immunomodulator (e.g., an activator or an inhibitor of an immune suppressor or an inhibitor of a checkpoint inhibitor), or treatment with a cancer cell or a mixture of antigens derived therefrom (e.g., treatment with antigens derived from a cancer cell line). Cancer immunotherapy includes a therapeutic treatment that stimulates or restores the ability of the immune system to fight cancer by inducing, enhancing or suppressing an immune response. Cancer immunotherapy results in targeting of an immune activity against a disease-specific antigen, either by increasing immune cell recognition of the target or by reducing disease-related immune suppression.

The term “tumor infiltrating T cell”, as used herein, generally refers to a T cell that infiltrates tumors. The tumor infiltrating T cells may appear naturally reactive to autologous tumor antigens. These cells can be found in the tumor stroma and/or within the tumor itself.

The term “IDO inhibitor”, as used herein, generally refers to an agent capable of inhibiting the activity of indoleamine 2,3-dioxygenase (IDO) and thereby reversing IDO-mediated immunosuppression. The IDO inhibitor may inhibit IDO1 and/or IDO2 (INDOL1). An IDO inhibitor may be a reversible or irreversible IDO inhibitor. “A reversible IDO inhibitor” is a compound that reversibly inhibits IDO enzyme activity either at the catalytic site or at a non-catalytic site and “an irreversible IDO inhibitor” is a compound that irreversibly destroys IDO enzyme activity by forming a covalent bond with the enzyme.

The term “immune checkpoint inhibitor”, as used herein, generally refers to any molecule that directly or indirectly inhibits, partially or completely, an immune checkpoint pathway. It is generally thought that immune checkpoint pathways function to turn on or off aspects of the immune system, particularly T cells, but also for instance myeloid cells, NK cells and B cells. Following activation of a T cell, a number of inhibitory receptors can be upregulated and present on the surface of the T cell in order to suppress the immune response at the appropriate time. Examples of immune checkpoint pathways include, without limitation, PD-1/PD-L1, CTLA-4/B7-1, TIM-3, LAG3, B7-H1, H4, HAVCR2, IDO1, CD276 and VTCN1, B7-H3, B7-H4, CD47, and KIR. For instance, non-limiting examples of immune checkpoint inhibitors or modulators include fully human monoclonal antibodies, such as BMS-936558/IDX-1106, BMS-s936559/IDX-1105, ipilimumab, and/or an antigen binding fragment or a derivative of any of the foregoing/Yervoy, tremelimumab, BMS-986016, Durvalumab, MEDI4736, Urelumab, CDX-1127, and Avelumab; humanized antibodies, such as CT-011, IV1K-3475, Hu5F9-G4, CC-90002, MBG453, TSR-022, and Atezolizumab; and fusion proteins, such as AMP-224 and TTI-621, and others. Other non-limiting examples of immune checkpoint modulators (agonists) include antibodies directed against e.g. CD40, OX40, GITR, CD137 (4-1 BB), CD27, ICOS, and TRAIL. In accordance with this invention, the one or more immune checkpoint modulator(s) may independently be a polypeptide or a polypeptide-encoding nucleic acid molecule; said polypeptide comprising a domain capable of binding the targeted immune checkpoint and/or inhibiting the binding of a ligand to said targeted immune checkpoint so as to exert an antagonist function (i.e. being capable of antagonizing an immune checkpoint-mediated inhibitory signal) or an agonist function (i.e. being capable of boosting an immune checkpoint-mediated stimulatory signal). Such one or more immune checkpoint modulator(s) can be independently selected from the group consisting of peptides (e.g. peptide ligands), soluble domains of natural receptors, RNAi, antisense molecules, antibodies and protein scaffolds. For example, the immune checkpoint modulator may be an antibody. In the context of the present disclosure, the immune check modulator antibody is used in the broadest sense and encompasses e.g. naturally occurring and engineered by man as well as full length antibodies or functional fragments or analogs thereof that are capable of binding the target immune checkpoint or epitope (thus retaining the target-binding portion). It can be of any origin, e.g. human, humanized, animal (e.g. rodent or camelid antibody) or chimeric. It may be of any isotype with a specific preference for an IgG1 or lgG4 isotype. In addition, it may be glycosylated or non-glycosylated. Standard assays to evaluate the binding ability of the antibodies toward immune checkpoints are known in the art, including for example, ELISAs, Western blots, RIAs and flow cytometry. The binding kinetics (e.g., binding affinity) of the antibodies also can be assessed by standard assays known in the art, such as by Biacore analysis. Where in the application reference is made to an immune checkpoint inhibitor, also an immune checkpoint modulator may be used, except in those cases where it is apparent from the context of the wording that this is not the case.

The term “exhaustion”, as used herein, generally refers to T cell exhaustion, which is a state of T-cell dysfunction that arises during many chronic infections and cancer. T cell exhaustion is characterized by poor T-cell effector function, sustained expression of inhibitory receptors and/or a transcriptional state distinct from that of functional effector or memory T-cells. Exhaustion prevents optimal control of infection and tumors. T-cell exhaustion may show a stepwise and progressive loss of T-cell functions. “Reversing exhaustion”, as used herein, generally refers to an activity or capability to restore at least some of the weakened or reduced anti-tumor activity of an exhausted T cell. Reversing exhaustion may also include preventing a T cell from being exhausted.

The term “T cell-mediated immune response”, as used herein, generally refers to an immune response influenced by modulation of T cell co-stimulation. Exemplary immune responses include T cell responses, e.g., cytokine production, and cellular cytotoxicity. In addition, T cell-mediated immune response also includes immune responses that are indirectly effected by T cell activation, e.g., antibody production (humoral responses) and activation of cytokine responsive cells, e.g., macrophages.

The term “tumor specific T cell”, as used herein, generally refers to T lymphocytes capable of specifically attacking and/or destroying tumor cells. For example, they may be endowed with a specific receptor (e.g., a T cell receptor) that can bind to an antigen present at the surface of a tumor cell, such as a tumor associated antigen. Each tumor specific T cell may recognize a single tumor antigen, and a group of tumor specific T cells may be endowed with a diversity of receptors targeted at a variety of tumor antigens.

The term “not substantially compete with”, as used herein, generally refers to that the binding of one molecule or agent to a target does not influence the binding of another molecule or agent to the same target in any significant way (e.g., to an extent by less than about 50%, by less than about 40%, by less than about 35%, by less than about 30%, by less than about 25%, by less than about 20%, by less than about 15%, by less than about 14%, by less than about 13%, by less than about 12%, by less than about 11%, by less than about 10%, by less than about 9%, by less than about 8%, by less than about 7%, by less than about 6%, by less than about 5%, by less than about 4%, by less than about 3%, by less than about 2%, by less than about 1%, by less than about 0.5%, or less), for e.g., as determined in an assay generally used to determine such binding (e.g., in an assay described in the Examples herein).

The term “antigen” or “Ag”, as used herein, generally refers to a molecule that provokes an immune response. This immune response may involve either antibody production, or the activation of specific immunologically-competent cells, or both. The person of ordinary skills in the art will understand that any macromolecule, including virtually all proteins or peptides, can serve as an antigen. Furthermore, antigens can be derived from recombinant or genomic DNA. A person of ordinary skills in the art will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. An antigen need not be encoded by a “gene”. It can be synthesized or can be derived from a biological sample, or might be macromolecule besides a polypeptide. Such a biological sample can include, but is not limited to a tissue sample, a tumor sample, a cell or a fluid with other biological components.

The term “anti-cancer” or “anti-tumor”, as used herein, generally refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer” or “anti-tumor” effect can also be manifested by the ability to prevent of the occurrence of cancer in the first place.

The term “hydrocarbyl”, as used herein, generally refers to a moiety consisting exclusively of hydrogen and carbon atoms; such a moiety may comprise an aliphatic and/or an aromatic moiety.

The moiety may comprise 1, 2, 3, 4, 5, 6, 7, 8, 9, 10, 11, 12, 13, 14, 15, 16, 17, 18, 19, 20, 30, 40, 50 or more carbon atoms. Examples of hydrocarbyl groups include without limitation alkyl such as C₁₋₆ alkyl (e.g. C₁, C₂, C₃ or C₄ alkyl, for example methyl, ethyl, propyl, isopropyl, n-butyl, sec-butyl or tert-butyl); C₁₋₆ alkyl substituted by aryl (e.g. benzyl) or by cycloalkyl (e.g cyclopropylmethyl); cycloalkyl (e.g. cyclopropyl, cyclobutyl, cyclopentyl or cyclohexyl); aryl (e.g. phenyl, naphthyl or fluorenyl) and the like.

The term “heterohydrocarbyl”, as used herein, generally refers to a hydrocarbyl group that optionally includes one or more heteroatoms. The heteroatoms may be any atom other than C, such as a O, S or N.

The term “alkenyl”, as used herein, generally refers to a straight or branched chain alkyl moiety having 2, 3, 4, 5, 6 or more carbon atoms and having, in addition, at least one double bond, of either E or Z stereochemistry where applicable. This term includes reference to groups such as ethenyl, 2-propenyl, 1-butenyl, 2-butenyl, 3-butenyl, 1-pentenyl, 2-pentenyl, 3-pentenyl, 1-hexenyl, 2-hexenyl and 3-hexenyl and the like.

The term “aryl”, as used herein, generally refers to an aromatic ring system comprising 6, 7, 8, 9, 10, 11, 12, 13, 14, 15, 16 or more ring carbon atoms. Aryl is often phenyl but may be a polycyclic ring system, having two or more rings, at least one of which is aromatic. This term includes reference to groups such as phenyl, naphthyl, fluorenyl, azulenyl, indenyl, anthryl and the like.

The term “prodrug”, as used herein, generally refers to compounds which are rapidly transformed in vivo to the parent compound, for example, by hydrolysis in blood.

The term “vaccine”, as used herein, generally refers to a preparation that provides active acquired immunity against a particular antigen (such as a tumor antigen, or an antigen of a microbe) or a tissue, cell, or organism comprising said antigen. Vaccines can be prophylactic (to prevent or ameliorate the effects of a future disease or disorder), or therapeutic (to treat a disease or disorder that has already occurred, such as cancer).

YTHDF1 Attenuating Agent

The YTHDF1 attenuating agent of the present application may comprise a compound.

Such a compound may be a macromolecule. A macromolecule may be a naturally occurring or chemically synthesized organic or inorganic molecule that is greater than or equal to about a 1000 Daltons to about or greater than 1, 2, 3, 5, 7, 10 or more trillion Daltons. A macromolecule may contain two or more monomeric subunits, or derivatives thereof, which are linked by a covalent bond, an ionic bond, or other chemical interactions, such as hydrogen bonding, ionic pairing, base pairing or pairing between charges formed by charge polarization. The monomeric subunits can be different from one another, or identical to one another, and, in some embodiments, can form a polymer. A macromolecule may also be a molecule that, regardless of whether it has more than one subunit and/or is a polymer, can form tertiary and/or quaternary structure. Examples of macromolecules include a polynucleotide, a nucleic acid molecule including DNA, RNA, including siRNA, snRNA, tRNA, antisense RNA, and ribozymes, peptide nucleic acid (PNA), a polypeptide, glycopeptides, a protein, a carbohydrate, or a lipid, or derivatives or combinations thereof, for example, a nucleic acid molecule containing a peptide nucleic acid portion or a glycoprotein, respectively. Examples of macromolecules further include macromolecular assemblies, for examples, viruses, virus particles, phages, viroids, prions and combinations and conjugates thereof.

Such a compound may be a small molecule. A small molecule may be a naturally occurring or chemically synthesized organic or inorganic molecule that is less than about 1000 Daltons, from about or at 1000 Daltons to about or at 950, 900, 850, 800, 750, 700, 650, 600, 550, 500, 450, 400, 375, 350, 325, 300, 275, 250, 225, 200, 175, 150, 125, 100, 75, 70, 65, 60, 55, 50, 45, 40, 35, 30, 25, 20, 15, 10, 5 or less Daltons. A small molecule may be any molecule that is not a macromolecule, such as a protein or nucleic acid. A “small molecule” can include a molecule containing two or more monomeric subunits, such as a dipeptide or dinucleotide.

Such a compound may comprise or be a polypeptide. In some cases, such a compound may comprise or be a nucleic acid molecule. For example, such a compound may comprise an antibody or a derivative thereof, an antibody-drug conjugate, and/or a fusion protein.

For example, the compound may be able to attenuate the activity of YTHDF1 protein. For example, the compound may directly or indirectly (e.g. through other molecules) bind to one or more residues of the YTHDF1 protein. Such binding may cause conformational changes to the structure and/or function of the YTHDF1 protein.

The compound may bind specifically to YTHDF1 (e.g., human YTHDF1), a fragment, or a derivative thereof. The YTHDF1 protein may comprise an amino acid sequence as set forth in SEQ ID NO: 1. In some cases, the YTHDF1, its fragment or derivative may at least comprise amino acid residues corresponding to residues N378, F382, W384, F480, and/or H528 of SEQ ID NO: 1. In some cases, the YTHDF1, its fragment or derivative may at least comprise amino acid residues corresponding to residues 372-392, 479-494 and/or 526-535 of SEQ ID NO: 1. In some cases, the compound may bind (e.g., specifically bind) to YTHDF1, or a fragment or derivative thereof, wherein the YTHDF1, its fragment or derivative may comprise an amino acid sequence as set forth in any of SEQ ID NOs: 1-3, 9-13 and 16-18. In some cases, the compound does not specifically bind (or, essentially does not bind) to a YTHDF1 or its fragment or derivative comprising an amino acid sequence as set forth in any of SEQ ID NOs: 4-8.

In some cases, the compound of the present application may bind to the YTHDF1, its fragment or derivative comprising an amino acid sequence as set forth in any of SEQ ID NOs: 4-8 with a K_{d} value of higher than about 10⁻⁶ M (e.g., higher than about 5×10⁻⁶ M, higher than about 10⁻⁵ M, higher than about 5×10⁻⁵ M, higher than about 10⁻⁴ M, higher than about 5×10⁻⁴ M, higher than about 10⁻³ M, higher than about 5×10⁻³ M, or higher). The K_{d} value may be determined using any method commonly used in the art, such as an Isothermal Titration Calorimetry (ITC) assay, a surface plasmon resonance (SPR) assay, and/or a microscale thermophoresis (MST) assay.

In some cases, the compound of the present application may bind to the YTHDF1, the fragment or the derivative thereof (e.g., as described in the present application, for example, those comprising/having an amino acid sequence as set forth in any of SEQ ID NOs: 1-3, 9-13 and 16-18) with a K_{d} value of less than about 10⁻⁵ M (e.g., less than about 9×10⁻⁶ M, less than about 8×10⁻⁶ M, less than about 7×10⁻⁶ M, less than about 6×10⁻⁶ M, less than about 5×10⁻⁶ M, less than about 4×10⁻⁶ M, less than about 3.5×10⁻⁶ M, less than about 3×10⁻⁶ M, less than about 2.5×10⁻⁶ M, less than about 2×10⁻⁶ M, less than about 1×10⁻⁶ M, less than about 5×10⁻⁷ M, less than about 2×10⁻⁷ M, less than about 10⁻⁷ M, less than about 5×10⁻⁸ M, less than about 2×10⁻⁸ M, less than about 10⁻⁸ M, less than about 5×10⁻⁹ M, less than about 4×10⁻⁹M, less than about 3×10⁻⁹M, less than about 2×10⁻⁹ M, or less than about 10⁻⁹ M). The K_{d} value may be determined using any method commonly used in the art, such as an Isothermal Titration Calorimetry (ITC) assay, a surface plasmon resonance (SPR) assay, and/or a microscale thermophoresis (MST) assay.

In some cases, the compound, when bound to YTHDF1, may bind (e.g., specifically bind) to at least one residue (e.g., at least 2, at least 3, at least 4, at least 5, at least 6, at least 7, at least 8, at least 9, at least 10, at least 15, at least 20, at least 25, at least 30, at least 35, at least 40, or more residues) corresponding to a residue selected from amino acid residues 372-392, 479-494 and 526-535 of SEQ ID NO: 1. In some cases, when the compound is bound to YTHDF1, it may bind (e.g., specifically bind) to multiple residues, and at least one (e.g., at least 2, at least 3, at least 4, at least 5, at least 6, at least 7, at least 8, at least 9, at least 10, at least 15, at least 20, at least 25, at least 30, at least 35, at least 40, or more residues) of the bound residues may be corresponding to a residue selected from amino acid residues 372-392, 479-494 and 526-535 of SEQ ID NO: 1.

In some cases, the compound, when bound to YTHDF1, may bind (e.g., specifically bind) to at least one (e.g., at least 2, at least 3, at least 4, or at least 5) residue corresponding to a residue selected from the following residues: N378, F382, W384, F480, and H528 of SEQ ID NO: 1.

The compound comprised by the YTHDF1 attenuating agent may be able to block binding of YTHDF1 (e.g., human YTHDF1), or a fragment or derivative thereof to m⁶A. The YTHDF1 protein may comprise an amino acid sequence as set forth in SEQ ID NO: 1. In some cases, the YTHDF1, its fragment or derivative may at least comprise amino acid residues corresponding to residues N378, F382, W384, F480, and/or H528 of SEQ ID NO: 1. In some cases, the YTHDF1, its fragment or derivative may at least comprise amino acid residues corresponding to residues 372-392, 479-494 and/or 526-535 of SEQ ID NO: 1.

In some cases, the compound may block binding of the YTHDF1, or the fragment or derivative thereof to m⁶A, wherein the YTHDF1, the fragment or the derivative may comprise an amino acid sequence as set forth in any of SEQ ID NOs: 1-3, 9-13 and 16-18.

In some cases, the compound does not significantly or substantially block binding of YTHDF1 or its fragment or derivative to m⁶A, wherein the YTHDF1, its fragment or the derivative may comprise an amino acid sequence as set forth in any of SEQ ID NOs: 4-8.

In some cases, the compound of the present application may block binding of the YTHDF1, its fragment or derivative to m⁶A with an IC₅₀ value of higher than about 7.5 μM (e.g., higher than about 8 μM, higher than about 8.5 μM, higher than about 9 μM, higher than about 9.5 μM, higher than about 10 M, higher than about 10.5 M, higher than about 11 μM, higher than about 11.5 μM, higher than about 12 μM, or higher), wherein the YTHDF1, its fragment or derivative may comprise an amino acid sequence as set forth in any of SEQ ID NOs: 4-8. The IC₅₀ value may be determined using any method commonly used in the art, such as a fluorescence polarization (FP) assay, and/or an AlphaScreen-based assay.

In some cases, the compound of the present application may block binding of the YTHDF1, its fragment or derivative to m⁶A with an IC₅₀ value of lower than about 7 μM (e.g., lower than about 6.5 μM, lower than about 6 μM, lower than about 5.5 μM, lower than about 5 μM, lower than about 4.5 μM, lower than about 4 μM, lower than about 3.5 μM, lower than about 3 μM, lower than about 2.5 μM, lower than about 2 μM, lower than about 1.5 μM, lower than about 1 μM, lower than about 0.9 μM, lower than about 0.8 M, lower than about 0.7 M, lower than about 0.6 M, lower than about 0.5 μM, lower than about 0.4 M, lower than about 0.3 M, lower than about 0.2 M, lower than about 0.1 μM, or lower), wherein the YTHDF1, its fragment or derivative may comprise an amino acid sequence as set forth in any of SEQ ID NOs: 1-3, 9-13 and 16-18. The IC₅₀ value may be determined using any method commonly used in the art, such as a fluorescence polarization (FP) assay, and/or an AlphaScreen-based assay.

In some cases, the compound comprised by the YTHDF1 attenuating agent does not substantially compete with m⁶A for binding to YTHDF1. For example, the binding of the compound to YTHDF1, its fragment or derivative is affected (e.g., decreased) by the addition of m⁶A by less than about 50%, by less than about 40%, by less than about 35%, by less than about 30%, by less than about 25%, by less than about 20%, by less than about 15%, by less than about 14%, by less than about 13%, by less than about 12%, by less than about 11%, by less than about 10%, by less than about 9%, by less than about 8%, by less than about 7%, by less than about 6%, by less than about 5%, by less than about 4%, by less than about 3%, by less than about 2%, by less than about 1%, by less than about 0.5%, or less), for e.g., as determined in an assay generally used to determine such binding (e.g., as shown in an AlphaScreen-based assay). For example, the binding of m⁶A to YTHDF1, its fragment or derivative is affected (e.g., decreased) by the addition of the compound of the present application by less than about 50%, by less than about 40%, by less than about 35%, by less than about 30%, by less than about 25%, by less than about 20%, by less than about 15%, by less than about 14%, by less than about 13%, by less than about 12%, by less than about 11%, by less than about 10%, by less than about 9%, by less than about 8%, by less than about 7%, by less than about 6%, by less than about 5%, by less than about 4%, by less than about 3%, by less than about 2%, by less than about 1%, by less than about 0.5%, or less), for e.g., as determined in an assay generally used to determine such binding (e.g., as shown in an AlphaScreen-based assay).

In some cases, the compound comprised by the YTHDF1 attenuating agent may be a salvianolic acid, such as a salvianolic acid A (SAA), a salvianolic acid C (SAC), a prodrug, a metabolite, a derivative thereof, or a pharmaceutically acceptable salt, ester, or amide of any of the foregoing, or any combinations thereof.

In some cases, the YTHDF1 attenuating agent may comprise a compound of Formula I, a prodrug, a metabolite, a derivative of the compound of Formula I, or a pharmaceutically acceptable salt, ester, or amide of any of the foregoing:

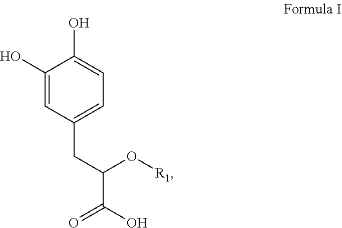

wherein R₁ may be selected from the group consisting of C₁₋₅₀ hydrocarbyl, C₁₋₅₀ substituted hydrocarbyl, C₁₋₅₀ heterohydrocarbyl and C₁₋₅₀ substituted heterohydrocarbyl.

In some cases, R₁ in Formula I may be (CO)—R₂, and R₂ may be an optionally substituted alkenyl. In some cases, R₂ may be CH═CH—R₃, and R₃ may be an optionally substituted aryl. In some cases, R₃ may be of Formula II

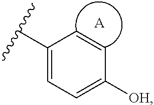

wherein A may be an optionally substituted furan, or

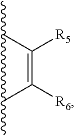

R₆ may be hydroxyl, and R₅ may be an optionally substituted alkenyl.

In some cases, in Formula II, A may be

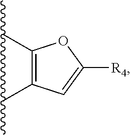

### and R₄ may be

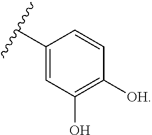 [SMILES: *c1ccc(O)c(O)c1]

In some cases, in Formula II, A may be

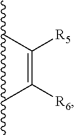

R₆ may be hydroxyl, R₅ may be CH═CH—R₇, and R₇ may be

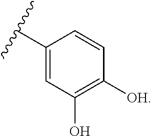 [SMILES: *c1ccc(O)c(O)c1]

In some cases, the compound comprised by the YTHDF1 attenuating agent of the present application may comprise at least two dihydroxyphenyl moieties.

In some cases, the compound comprised by the YTHDF1 attenuating agent may comprise at least three dihydroxyphenyl moieties.

In some cases, the YTHDF1 attenuating agent may comprise a compound of Formula III, a prodrug, a metabolite, a derivative of the compound of Formula III, or a pharmaceutically acceptable salt, ester, or amide of any of the foregoing:

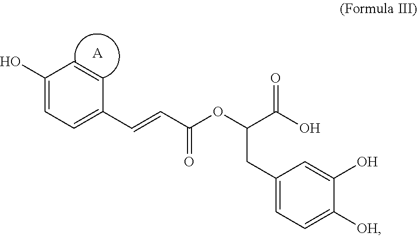

wherein A may be an optionally substituted furan, or

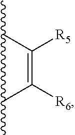

R₆ may be hydroxyl, and R₅ may be an optionally substituted alkenyl.

In some case, in Formula III, A may be

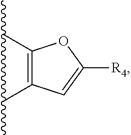

### and R₄ may be

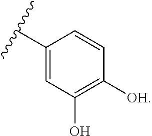 [SMILES: *c1ccc(O)c(O)c1]

In some cases, in Formula III, A may be

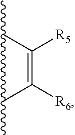

R₆ may be hydroxyl, R₅ may be CH═CH—R₇, and R₇ may be

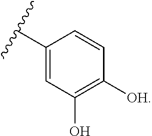 [SMILES: *c1ccc(O)c(O)c1]

In some cases, the YTHDF1 attenuating agent may comprise any of the following compounds, a prodrug, a metabolite, a derivative of any of the following compounds, or a pharmaceutically acceptable salt, ester, or amide of any of the foregoing:

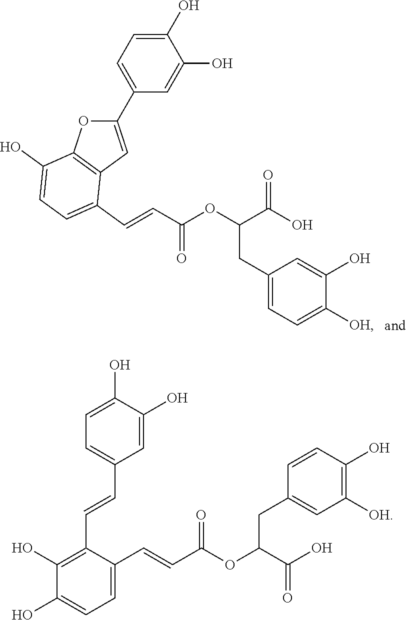 [SMILES: O=C(/C=C/c1ccc(O)c2oc(-c3ccc(O)c(O)c3)cc12)OC(Cc1ccc(O)c(O)c1)C(=O)O ; "and" ; O=C(/C=C/c1ccc(O)c(O)c1/C=C/c1ccc(O)c(O)c1)OC(Cc1ccc(O)c(O)c1)C(=O)O]

In some cases, the YTHDF1 attenuating agent may comprise any of the following compounds, a prodrug, a metabolite, a derivative of any of the following compounds, or a pharmaceutically acceptable salt, ester, or amide of any of the foregoing: (E)-3-(3,4-dihydroxyphenyl)-2-((3-(2-(3,4-dihydroxyphenyl)-7-hydroxybenzofuran-4-yl)acryloyl)oxy)propanoic acid, and 3-(3,4-dihydroxyphenyl)-2-(((E)-3-(2-((E)-3,4-dihydroxystyryl)-3,4-dihydroxyphenyl)acryloyl)oxy)propanoic acid.

In some cases, the YTHDF1 attenuating agent may comprise any of the following compounds, a prodrug, a metabolite, a derivative of any of the following compounds, or a pharmaceutically acceptable salt, ester, or amide of any of the foregoing:

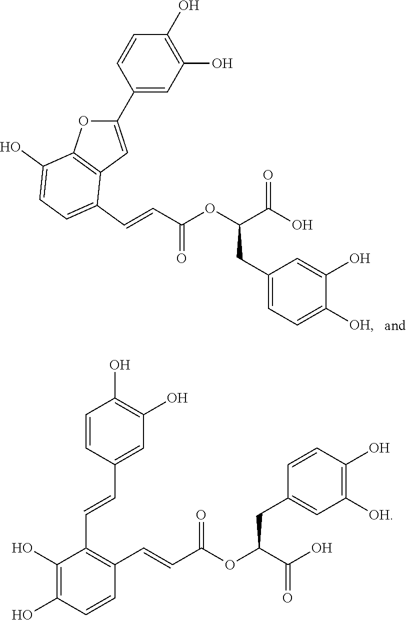 [SMILES: O=C(/C=C/c1ccc(O)c2oc(-c3ccc(O)c(O)c3)cc12)O[C@H](Cc1ccc(O)c(O)c1)C(=O)O ; "and" ; O=C(/C=C/c1ccc(O)c(O)c1/C=C/c1ccc(O)c(O)c1)O[C@@H](Cc1ccc(O)c(O)c1)C(=O)O]

In some cases, the YTHDF1 attenuating agent may comprise any of the following compounds, a prodrug, a metabolite, a derivative of any of the following compounds, or a pharmaceutically acceptable salt, ester, or amide of any of the foregoing: (R,E)-3-(3,4-dihydroxyphenyl)-2-((3-(2-(3,4-dihydroxyphenyl)-7-hydroxybenzofuran-4-yl)acryloyl)oxy)propanoic acid, and (S)-3-(3,4-dihydroxyphenyl)-2-(((E)-3-(2-((E)-3,4-dihydroxystyryl)-3,4-dihydroxyphenyl)acryloyl)oxy)propanoic acid.

In some cases, the YTHDF1 attenuating agent may comprise any of the following compounds, a prodrug, a metabolite, a derivative of any of the following compounds, or a pharmaceutically acceptable salt, ester, or amide of any of the foregoing:

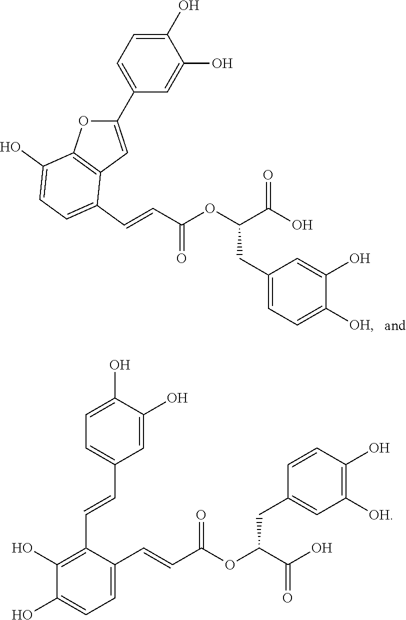 [SMILES: O=C(/C=C/c1ccc(O)c2oc(-c3ccc(O)c(O)c3)cc12)O[C@@H](Cc1ccc(O)c(O)c1)C(=O)O ; "and" ; O=C(/C=C/c1ccc(O)c(O)c1/C=C/c1ccc(O)c(O)c1)O[C@H](Cc1ccc(O)c(O)c1)C(=O)O]

In some cases, the YTHDF1 attenuating agent may comprise any of the following compounds, a prodrug, a metabolite, a derivative of any of the following compounds, or a pharmaceutically acceptable salt, ester, or amide of any of the foregoing: (S,E)-3-(3,4-dihydroxyphenyl)-2-((3-(2-(3,4-dihydroxyphenyl)-7-hydroxybenzofuran-4-yl)acryloyl)oxy)propanoic acid, and (R)-3-(3,4-dihydroxyphenyl)-2-(((E)-3-(2-((E)-3,4-dihydroxystyryl)-3,4-dihydroxyphenyl)acryloyl)oxy)propanoic acid.

In some cases, the YTHDF1 attenuating agent may be achiral or chiral, if the YTHDF1 attenuating is chiral, it may have one or more chiral centers and may be a single (R) or (S) enantiomer or a mixture of (R) and (S) enantiomers.

In some cases, the compound comprised by the YTHDF1 attenuating agent may be derived from a plant. For example, the compound may be provided in a plant extract, e.g., as part of a plant extract. For example, it may be derived from the Salvia species and active constituents thereof.

The compound may be chemically manufactured (e.g. from oleochemicals), biochemically produced (e.g. in fermentation processes), or may be obtained from plant material, optionally followed by subsequently chemical modification. For example, the compound may be (bio)chemically manufactured by esterifying 3-(3,4-dihydroxyphenyl)lactic acid with a carboxylic acid.

In some cases, the compound may be isolated from a plant material, such as the roots of plants. For example, the plant may belong to the genus Salvia, e.g., Salvia miltiorrhiza, Salvia cavaleriei, Salvia fluva, Salvia chinensis, Salvia bowleyana, Salvia prionitis, Salvia officialis, Salvia deserta and/or Salvia yunnanensis. In some cases, the compound is obtained from Salvia milthiorrhiza (Danshen).

Modified Immune Cells

The present application provides modified immune cells (e.g., APCs, such as DCs). The present application also provides methods for modifying immune cells (e.g., APCs, such as DCs).

The immune cells may be APCs, such as DCs. The APCs (e.g., DCs) may be derived from bone marrow and/or from lymph node of a subject. The DCs may comprise one or more of the following: resident CD11b⁺ cells (e.g., CD11b⁺ DCs), resident CD8α⁺ cells (e.g., CD8α⁺ DCs), migratory CD11b⁺ cells (e.g., CD11b⁺ DCs), CD11c⁺ cells (e.g., CD11c+ DCs) and migratory CD103⁺ cells (e.g., CD103⁺ DCs).

The modified APCs (e.g., modified DCs) may exhibit better ability in cross-priming T cells than corresponding unmodified control APCs (e.g., corresponding unmodified control DCs).

The APCs (e.g., DCs) may comprise or express one or more tumor specific antigens (e.g., a tumor/cancer associated antigen as provided in the present application). In some cases, the APCs (e.g., DCs) may be co-cultured or treated with a simulating agent (such as FLT3L).

In some cases, the immune cell may be an APC (e.g., a DC) obtained from a subject (such as a cancer patient), in some cases, the immune cell (e.g., APC, such as DC) may be isolated from a tumor tissue.

The immune cell (e.g., APC, such as DC) may have been modified with a compound or a YTHDF1 attenuating agent of the present application.

For example, in a population of immune cells (e.g., APC, such as DC), one or more cells may have been modified with a compound or a YTHDF1 attenuating agent of the present application. In some cases, the modified immune cells (e.g., mAPCs, such as mDCs) of the present application may comprise a compound or a YTHDF1 attenuating agent of the present application.

In some cases, the compound or the YTHDF1 attenuating agent of the present application may be allowed to be in contact with the immune cells (e.g., mAPCs, such as mDCs) for a period of time (e.g., at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, or longer) sufficient to result in an reduced expression and/or activity of YTHDF1, for example, the compound or agent may be administered in a medium for culturing the immune cells (e.g., mAPCs, such as mDCs).

The compound or the YTHDF1 attenuating agent of the present application may be administered at a concentration of e.g. at least 1 μM, at least 2 μM, at least 3 μM, at least 4 μM, at least 5 μM, at least 6 μM, at least 7 μM, at least 8 μM, at least 9 μM, at least 10 μM, at least 11 μM, at least 12 μM, or higher.

In some cases, the compound or the YTHDF1 attenuating agent of the present application is not applied directly to the immune cell (e.g., APCs, or DCs) itself, instead, the immune cell (e.g, APCs, such as DCs) may be derived from (e.g., differentiated from, as a progeny of, etc.) a cell (e.g., a progenitor of an immune cell) or an organism that has been subjected to the compound or the YTHDF1 attenuating agent of the present application.

The immune cells may be human cells, such as human APCs (e.g., DCs).

In some cases, prior to expansion or other modification, a source of the cells, e.g., the immune cell (such as the APCs, e.g., DCs) or a progenitor cell thereof may be obtained from a subject. The term “subject” herein is intended to include living organisms in which an immune response can be elicited (e.g., mammals). Examples of subjects include humans, monkeys, chimpanzees, dogs, cats, mice, rats, and transgenic species thereof. The immune cells or progenitors thereof may be obtained from a number of sources, including peripheral blood mononuclear cells, bone marrow, lymph node tissue, cord blood, thymus tissue, tissue from a site of infection, ascites, pleural effusion, spleen tissue, and/or tumors.

Compositions

A composition of the present application may comprise a YTHDF1 attenuating agent of the present application, and/or a mAPC of the present application. In some cases, the composition may further comprise an additional/second active ingredient of the present application.

In some cases, the composition may be a vaccine composition.

The composition of the present application may comprise one or more pharmaceutically acceptable excipients. Such pharmaceutically acceptable excipient may include any inactive material that is combined with one or more active ingredient (e.g., the modified cell or attenuating agent) of the present application.

For example, the pharmaceutically acceptable excipient may include one or more of the following: a solvent, a penetration enhancing agent, an antioxidant, a thickener, an ointment base, a protective, an adsorbent, a demulcent, an emollient, a preservative, a moisturizer, a buffer, an adjuvant, a bioavailability enhancer, a carrier, a glidant, a sweetening agent, a diluent, a dye/colorant, a flavor enhancer, a solubilizer (including surfactants), a wetting agent, a dispersing agent, a suspending agent, a stabilizer and/or an isotonic agent.

In some cases, the composition may comprise one or more adjuvant to enhance or increase an immune response associated with administration of the composition.

Additional/Second Active Ingredient

The compound, the YTHDF1 attenuating agent, the cells (e.g., mAPCs, mDCs) and/or the composition of the present application may further comprise, and/or may be used in combination with an additional/second active ingredient.

In some cases, the compound, the YTHDF1 attenuating agent, the cells (e.g., mAPCs, mDCs) and/or the composition of the present application may be administered to a subject that has received, is receiving, and/or will receive an additional/second active ingredient.

The additional active ingredient or therapy may be administered prior to, concurrent with, and/or after the administration of the YTHDF1 attenuating agent, the cells (e.g., mAPCs, mDCs) and/or the composition of the present application.

The additional active ingredient may be an anti-cancer agent. For example, the additional active ingredient may comprise a cancer immunotherapy. In some cases, the additional active ingredient may comprise an immune checkpoint attenuating agent (e.g., an immune checkpoint inhibitor). In some cases, the additional active ingredient may comprise an agent selected from the group consisting of: an anti-PD-L1 antibody or an antigen binding portion thereof, an anti-PD-1 antibody or an antigen binding portion thereof, an anti-CTLA-4 antibody or an antigen binding portion thereof, and an IDO attenuating agent.

For example, the additional active ingredient may comprise pembrolizumab, nivolumab, cemiplimab, atezolizumab, avelumab, durvalumab, ipilimumab, and/or an antigen binding fragment or a derivative of any of the foregoing. For example, the additional active ingredient may comprise an antibody (including an antigen binding part thereof) capable of competing with pembrolizumab, nivolumab, cemiplimab, atezolizumab, avelumab, durvalumab, and/or ipilimumab for binding to the corresponding antigen (e.g., PD-1, PD-L1, or CTLA-4, respectively). In some cases, the additional active ingredient may comprise a HCDR3 of any of pembrolizumab, nivolumab, cemiplimab, atezolizumab, avelumab, durvalumab, and/or ipilimumab. In some cases, the additional active ingredient may comprise a LCDR3 of any of pembrolizumab, nivolumab, cemiplimab, atezolizumab, avelumab, durvalumab, and/or ipilimumab. In some cases, the additional active ingredient may comprise a HCDR2 of any of pembrolizumab, nivolumab, cemiplimab, atezolizumab, avelumab, durvalumab, and/or ipilimumab. In some cases, the additional active ingredient may comprise a LCDR2 of any of pembrolizumab, nivolumab, cemiplimab, atezolizumab, avelumab, durvalumab, and/or ipilimumab. In some cases, the additional active ingredient may comprise a HCDR1 of any of pembrolizumab, nivolumab, cemiplimab, atezolizumab, avelumab, durvalumab, and/or ipilimumab. In some cases, the additional active ingredient may comprise a LCDR1 of any of pembrolizumab, nivolumab, cemiplimab, atezolizumab, avelumab, durvalumab, and/or ipilimumab. In some cases, the additional active ingredient may comprise a HCDR3, HCDR2, and HCDR1 of any of pembrolizumab, nivolumab, cemiplimab, atezolizumab, avelumab, durvalumab, and/or ipilimumab. In some cases, the additional active ingredient may comprise a LCDR1, LCDR2, and LCDR3 of any of pembrolizumab, nivolumab, cemiplimab, atezolizumab, avelumab, durvalumab, and/or ipilimumab. In some cases, the additional active ingredient may comprise a HCDR1, HCDR2, HCDR3, LCDR1, LCDR2, and LCDR3 of any of pembrolizumab, nivolumab, cemiplimab, atezolizumab, avelumab, durvalumab, and/or ipilimumab. In some cases, the additional active ingredient may comprise a heavy chain variable region of any of pembrolizumab, nivolumab, cemiplimab, atezolizumab, avelumab, durvalumab, and/or ipilimumab. In some cases, the additional active ingredient may comprise a light chain variable region of any of pembrolizumab, nivolumab, cemiplimab, atezolizumab, avelumab, durvalumab, and/or ipilimumab. In some cases, the additional active ingredient may comprise a heavy chain variable region and a light chain variable region of any of pembrolizumab, nivolumab, cemiplimab, atezolizumab, avelumab, durvalumab, and/or ipilimumab.

Inhibition Methods

The present application provides methods for inhibiting and/or attenuating the activity of YTHDF1.

The present application further provides methods for activating an immune cell (e.g., an APC, such as a DC), generating an immune cell having enhanced anti-tumor activity, preventing and/or reversing exhaustion of an immune cell (such as immune effector cells, e.g., T cells), increasing and/or improving proliferation and/or activity of immune cells (e.g., immune effector cells, such as T cells, for example, tumor infiltrating T cells), increasing and/or improving proliferation and/or activity of tumor specific immune cells (e.g., immune effector cells, such as T cells), enhancing cytokine production of immune cells (such as T cells), and/or inhibiting tumor growth, inhibiting the proliferation of tumor cells, and/or killing tumor cells.

Such method may comprise a step of administering the YTHDF1 attenuating agent, the cells (e.g., mAPCs, mDCs) and/or the composition of the present application.

For example, the method may comprise contacting YTHDF1, or a target cell comprising YTHDF1 (e.g., immune cells, such as APCs and/or T cells) with the YTHDF1 attenuating agent, the cells (e.g., mAPCs, mDCs) and/or the composition of the present application. The contacting may be done ex vivo. In some cases, the contacting may be done in vivo.

In some cases, the method may comprise introducing into said cells (e.g., immune cells, such as APCs and/or T cells) the YTHDF1 attenuating agent and/or the composition of the present application. The introducing may be done ex vivo. In some cases, the introducing may be done in vivo. In some cases, the introducing may be done ex vivo.

Inhibitor Screening Method and Kit

The present application provides a method for determining whether or not a candidate agent is a YTHDF1 attenuating agent.

The method may comprise contacting the candidate agent with a YTHDF1 mutant.

The YTHDF1 mutant may comprise one or more amino acid substitution, deletion and/or addition at one or more residues corresponding to a residue selected from residues 372-392, 479-494 and 526-535 of SEQ ID NO: 1. In some cases, the YTHDF1 mutant may comprise one or more amino acid substitution, deletion and/or addition at one or more residues corresponding to a residue selected from residues N378, F382, W384, F480, and H528 of SEQ ID NO: 1. In some cases, the YTHDF1 mutant may comprise one or more amino acid substitutions corresponding to the following amino acid substitutions: N378A, F382A, W384A, F480A and H528A, based on the amino acid sequence as set forth in SEQ ID NO: 1. In some cases, the YTHDF1 mutant may comprise an amino acid sequence as set forth in any of SEQ ID NOs: 4-8.

The method may further comprise determining whether or not the candidate agent specifically binds to the YTHDF1 mutant of the present application.

If the candidate agent specifically binds to the YTHDF1 mutant of the present application, then, the candidate agent may not be a YTHDF1 attenuating agent.

The method may further comprise contacting the candidate agent with a control YTHDF1, it's fragment or derivative, and determining whether or not the candidate agent specifically binds to the control YTHDF1, its fragment or derivative. In some cases, the control YTHDF1, its fragment or derivative may at least comprise amino acid residues corresponding to residues N378, F382, W384, F480, and/or H528 of SEQ ID NO: 1. In some cases, the control YTHDF1, its fragment or derivative may at least comprise amino acid residues corresponding to residues 372-392, 479-494 and/or 526-535 of SEQ ID NO: 1. In some cases, the control YTHDF1, its fragment or derivative may comprise an amino acid sequence as set forth in any of SEQ ID NOs: 1-3, 9-13 and 16-18.

In some cases, the method may further comprise determining whether or not the candidate agent specifically binds to the control YTHDF1, its fragment or derivative.

If the candidate agent does not specifically bind to the control YTHDF1, its fragment or derivative of the present application, then, the candidate agent may not be a YTHDF1 attenuating agent.

If the candidate agent specifically binds to the control YTHDF1, its fragment or derivative of the present application, but does not specifically bind to the YTHDF1 mutant of the present application, then, the candidate agent may be considered as a potential YTHDF1 attenuating agent.

In one aspect, the present application provides a YTHDF1 mutant (such as a YTHDF1 mutant described in the present application), e.g., for screening and/or determining an activity of a candidate agent to attenuate YTHDF1.

In one aspect, the present application provides a kit comprising a YTHDF1 mutant of the present application. The kit may be used e.g., for screening and/or determining an activity of a candidate agent to attenuate YTHDF1.

The kit may further comprise additional agents. For example, the kit may comprise a control YTHDF1, its fragment or derivative of the present application.

In some cases, the kit may further comprise a buffer, or agents useful in an assay (e.g, an Isothermal Titration Calorimetry (ITC) assay, the surface plasmon resonance (SPR) assay, and/or a microscale thermophoresis (MST) assay) for determining binding affinity of the candidate agent.

Antigen

The compound, the YTHDF1 attenuating agent, the cells (e.g., mAPCs, mDCs) and/or the composition of the present application may be used for treating a disease, disorder or condition associated with an expression of an antigen in a subject in need thereof, and/or may be used for stimulating a T cell-mediated immune response to a an antigen (e.g., tumor antigen) in a subject in need thereof.

Further, the compound, the YTHDF1 attenuating agent, the cells (e.g., mAPCs, mDCs) and/or the composition of the present application may be used in combination with an additional/second active ingredient, the additional/second active ingredient may cause an increase of one or more antigens (e.g., tumor antigens) in a subject receiving it.

The antigen may be any molecule capable of provoking an immune response, e.g., in a human subject. This immune response may involve either antibody production, or the activation of specific immunologically-competent cells, or both. Any macromolecule, including virtually all proteins or peptides, may serve as an antigen. The antigen may be derived from and/or present in a biological sample. Such a biological sample can include, but is not limited to a tissue sample, a tumor sample, a cell or a fluid with other biological components.

In the present application, cancer associated antigens or tumor antigens may be expressed on the surface of cancer cells. In some cases, the cancer associated antigens themselves may be intracellular, however, a fragment of such antigen (peptide) may be presented on the surface of the cancer cells by MHC (major histocompatibility complex).

Examples of cancer/tumor associated antigens may include e.g., EGFR, HER2/neu, HER3, HER4, Ep-CAM, CEA, TrAIL, TRAIL receptor 1, TRAIL receptor 2, lymphotoxin-beta receptor, CCR4, CD19, CD20, CD22, CD28, CD33, CD40, CD80, CSF-1R, CTLA-4, fibroblast activation protein (FAP), hepsin, chondroitin proteoglycan sulfate associated with melanoma (MCSP), prostate specific membrane antigen (PSMA), VEGF receptor 1, VEGF receptor 2, IGF-1R, TSLP-R, TIE-1, TIE-2, TNF-alpha, and similar weak apoptosis inducer to TNF (TWEAK), IL-1R.

In some cases, examples of cancer/tumor associated antigens may include e.g. CEA, gp100, the MAGE family of proteins, DAGE, GAGE, RAGE, NY-ESO 1, Melan-A/MART 1, TRP-1, TRP-2, tyrosinase, HER-2/neu, MUC-1, p53, KSA, PSA, PSMA, and/or fragments and modified versions thereof.

Enhancing Anti-Tumor Immunity

The compound, the YTHDF1 attenuating agent, the cells (e.g., mAPCs, mDCs), the methods, and/or the composition of the present application may be used for activating an immune cell, and/or for enhancing an immune response, e.g. an anti-tumor immune response.

For example, an activated immune cell may have increased ability (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 100%, at least about 1.5 folds, at least about 2 folds, at least about 2.5 folds, at least about 3 folds, at least about 3.5 folds, at least about 4 folds, at least about 4.5 folds, or more) to kill tumor cells or control tumor growth in vivo.

In some cases, in a population of immune cells, an increased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 100%, at least about 1.5 folds, at least about 2 folds, at least about 2.5 folds, at least about 3 folds, at least about 3.5 folds, at least about 4 folds, at least about 4.5 folds, or more) proliferation of CD4⁺ T cells may be observed. In some cases, in a population of immune cells, an increased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 100%, at least about 1.5 folds, at least about 2 folds, at least about 2.5 folds, at least about 3 folds, at least about 3.5 folds, at least about 4 folds, at least about 4.5 folds, or more) proliferation of CD8⁺ T cells may be observed.

In some cases, an enhanced anti-tumor immune response may be revealed by an increase (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 100%, at least about 1.5 folds, at least about 2 folds, at least about 2.5 folds, at least about 3 folds, at least about 3.5 folds, at least about 4 folds, at least about 4.5 folds, or more) of the number of CD8⁺ cytotoxic T cells in or surrounding the site of a tumor.

In some cases, an enhanced anti-tumor immune response may be revealed by an increase (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 100%, at least about 1.5 folds, at least about 2 folds, at least about 2.5 folds, at least about 3 folds, at least about 3.5 folds, at least about 4 folds, at least about 4.5 folds, or more) of the number of tumor infiltrating CD8⁺ T cells.

In some cases, increased activity of an immune cell (e.g. T cell) may be revealed by increased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 100%, at least about 1.5 folds, at least about 2 folds, at least about 2.5 folds, at least about 3 folds, at least about 3.5 folds, at least about 4 folds, at least about 4.5 folds, or more) cytokine (e.g., IFN-γ, and/or IL-2) and/or Granzyme B production by the immune cells.

In some cases, an increased activity of immune cells or an enhanced immune response may be revealed by delayed and/or reversed (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 100%, at least about 1.5 folds, at least about 2 folds, at least about 2.5 folds, at least about 3 folds, at least about 3.5 folds, at least about 4 folds, at least about 4.5 folds, or more) exhaustion of an immune cells, such as delayed and/or reversed (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 100%, at least about 1.5 folds, at least about 2 folds, at least about 2.5 folds, at least about 3 folds, at least about 3.5 folds, at least about 4 folds, at least about 4.5 folds, or more) exhaustion of CD8⁺ T cells.

For example, the increased activity of immune cells or an enhanced immune response may be revealed by an increased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 100%, at least about 1.5 folds, at least about 2 folds, at least about 2.5 folds, at least about 3 folds, at least about 3.5 folds, at least about 4 folds, at least about 4.5 folds, or more) expression of CXCR5. The increased expression may either be characterized by an increased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 100%, at least about 1.5 folds, at least about 2 folds, at least about 2.5 folds, at least about 3 folds, at least about 3.5 folds, at least about 4 folds, at least about 4.5 folds, or more) amount/level of CXCR5 in/on the cells, or be characterized by an increased number/percentage (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 100%, at least about 1.5 folds, at least about 2 folds, at least about 2.5 folds, at least about 3 folds, at least about 3.5 folds, at least about 4 folds, at least about 4.5 folds, or more) of cells that express CXCR5 among a population of immune cells (e.g., a population of immune effector cells, such as a population of T cells).

In some cases, an increased activity of immune cells or an enhanced immune response may be revealed by a decreased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 100%, at least about 1.5 folds, at least about 2 folds, at least about 2.5 folds, at least about 3 folds, at least about 3.5 folds, at least about 4 folds, at least about 4.5 folds, or more) expression of PD-1. The decreased expression may either be characterized by a decreased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 100%, at least about 1.5 folds, at least about 2 folds, at least about 2.5 folds, at least about 3 folds, at least about 3.5 folds, at least about 4 folds, at least about 4.5 folds, or more) amount/level of PD-1 in/on the cells, or be characterized by a decreased number/percentage (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 100%, at least about 1.5 folds, at least about 2 folds, at least about 2.5 folds, at least about 3 folds, at least about 3.5 folds, at least about 4 folds, at least about 4.5 folds, or more) of cells that express PD-1 among a population of immune cells (e.g., a population of immune effector cells, such as a population of T cells).

In some cases, an increased activity of immune cells or an enhanced immune response may be revealed by a decreased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 100%, at least about 1.5 folds, at least about 2 folds, at least about 2.5 folds, at least about 3 folds, at least about 3.5 folds, at least about 4 folds, at least about 4.5 folds, or more) expression of Tim3. The decreased expression may either be characterized by a decreased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 100%, at least about 1.5 folds, at least about 2 folds, at least about 2.5 folds, at least about 3 folds, at least about 3.5 folds, at least about 4 folds, at least about 4.5 folds, or more) amount/level of Tim3 in/on the cells, or be characterized by a decreased (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 100%, at least about 1.5 folds, at least about 2 folds, at least about 2.5 folds, at least about 3 folds, at least about 3.5 folds, at least about 4 folds, at least about 4.5 folds, or more) number/percentage of cells that express Tim3 among a population of immune cells (e.g., a population of immune effector cells, such as a population of T cells).

In some cases, an increased activity of immune cells or an enhanced immune response may be revealed by an decreased number and/or percentage (e.g., by at least about 1%, at least about 2%, at least about 3%, at least about 4%, at least about 5%, at least about 8%, at least about 10% at least about 15%, at least about 16%, at least about 17%, at least about 18%, at least about 19%, at least about 20%, at least about 25%, at least about 30%, at least about 35%, at least about 40%, at least about 45%, at least about 50%, at least about 100%, at least about 1.5 folds, at least about 2 folds, at least about 2.5 folds, at least about 3 folds, at least about 3.5 folds, at least about 4 folds, at least about 4.5 folds, or more) of PD-1⁺Tim3⁺ cells within a population of immune cells (e.g., a population of immune effector cells, such as a population of T cells).

Disease, Disorder or Condition

The compound, the YTHDF1 attenuating agent, the cells (e.g., mAPCs, mDCs), the methods, and/or the composition of the present application may be used for treating a disease, disorder or condition, such as a disease, disorder or condition associated with an expression of an antigen (e.g., a cancer/tumor associated antigen described herein) in a subject in need thereof.

For example, the disease, disorder or condition may be cancer.

In some cases, the cancer may be selected from the group consisting of a hematological cancer, a lymphoma, and a solid tumor.

In some embodiments, the cancer is selected from the group consisting of melanoma, breast cancer, lung cancer, ovarian cancer, brain cancer, liver cancer, cervical cancer, colon cancer, colorectal cancer, renal cancer, skin cancer, head & neck cancer, bone cancer, esophageal cancer, bladder cancer, uterine cancer, lymphatic cancer, stomach cancer, pancreatic cancer, testicular cancer, lymphoma, and leukemia.

Subject

The compound, the YTHDF1 attenuating agent, the cells (e.g., mAPCs, mDCs), the methods, and/or the composition of the present application may be administered to a subject (e.g., a human being) in need thereof.

In some cases, the subject may be a cancer patient. For example, the subject may be a patient of a cancer selected from the group consisting of a hematological cancer, a lymphoma, and a solid tumor. In some cases, the subject may be a patient of a cancer selected from the group consisting of melanoma, colon cancer, pancreatic cancer, breast cancer and lung cancer.

In some cases, the subject may have received, is receiving, and/or will receive an additional therapy. The additional therapy may be an anti-cancer treatment.

In some cases, the anti-cancer treatment may comprise a cancer immunotherapy. For example, the anti-cancer treatment may comprise or is an immune checkpoint attenuating agent. In some cases, the anti-cancer treatment may comprise an agent selected from the group consisting of: an anti-PD-L1 antibody or an antigen binding portion thereof, an anti-PD-1 antibody or an antigen binding portion thereof, an anti-CTLA-4 antibody or an antigen binding portion thereof, and an IDO attenuating agent.

In some cases, the anti-cancer treatment may comprise pembrolizumab, nivolumab, cemiplimab, atezolizumab, avelumab, durvalumab, ipilimumab, and/or an antigen binding fragment or a derivative of any of the foregoing.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like. As for the experimental results (e.g., in a two-sided unpaired Student's t-test), * indicates p<0.05, ** indicates p<0.01, *** indicates p<0.001, *** indicates p<0.0001, and n.s. means not significant.

Materials and Methods

The following materials and methods were employed in the Examples of the present application.

Protein Expression and purification

The protein YTHDF1 (361-559) (SEQ ID NO: 2) and its mutants (SEQ ID NOs: 4-13) were cloned into pGEx-6P-1 vector (obtained from YouBio Co, Ltd, Catalog NO: VT1258), and the His-YTHDF1 (361-559) (SEQ ID NO: 3) used in AlphaScreen Assay was cloned into modified pET28a vector (obtained from YouBio Co, Ltd, Catalog NO: VT1207). The vectors were transformed into E. coli BL21 (DE3) cells and cultured at 37° C. When OD value reached 0.6˜0.8, 1 mM Isopropyl-β-D-thiogalactopyranoside was added to overexpress the protein at 16° C. overnight. YTHDF1(361-559) and the mutants were purified by glutathione affinity chromatography (GSTrap FF, GE Healthcare) firstly and were incubated with PPase at 4° C. overnight to remove the GST tag. Then, the proteins were further purified via cation exchange (HiTrap SP, GE Healthcare) and finally through a Superdex 75 10/300 column (GE Healthcare). At last, the purified YTHDF1(361-559) and the mutants were kept in buffer which contains 20 mM Hepes (pH 7.4) and 200 mM NaCl. For His-YTHDF1 (361-559), the protein was purified via Ni-NTA chromatography (HisTrap FF, GE Healthcare), followed by cation exchange and Superdex 75 10/300 column in sequence. And the obtained protein was kept in the buffer with the same ingredients.

Fluorescence Polarization (FP) Assay

All of the YTHDF1 (361-559), the 5′-FAM labeled m⁶A-containing mRNA (5′-FAM-UUCUUCUGUGG (m⁶A) CUGUG-3′, SEQ ID NO: 14) and the candidate compounds were diluted with assay buffer (20 mM Hepes (pH 7.4), 50 mM NaCl, 0.01% (v/v) tween 20, 5% (v/v) glycerol). For high throughput screening (HTS), 1.25 μM YTHDF1 (361-559) was incubated with 80 μM candidate compounds in black 384 plate (Corning, 3575) at room temperature for 30 minutes. Then 40 nM 5′-FAM labeled m⁶A-containing mRNA was added into the mixture and incubated at 4° C. for 1 hour. Unlabeled m⁶A-containing mRNA was used as the positive control and 40 nM 5′-FAM labeled m⁶A-containing mRNA alone was used to adjust the gain factor. At last, the mixtures were measured by Envision Readers (PerkinElmer).

As for activity tests, 1.25 μM YTHDF1 (361-559) was incubated with candidate compounds diluted to concentrations as indicated for 30 minutes. The subsequent steps were similar to that of HTS. And an equal amount of DMSO was used as the negative control.

AlphaScreen Assay

Compound (e.g., SAA or SAC) was diluted from 200 μM in a two-fold gradient manner with assay buffer (20 mM Hepes (pH 7.4), 150 mM NaCl, 1 mg/ml BSA, 0.01% (v/v) TritonX-100). Next, 100 nM His-YTHDF1 (361-559) was incubated with SAA or SAC in assay buffer at room temperature for 30 minutes. Then 10 nM biotinylated m⁶A-containing mRNA (5′-biotin-UUCUUCUGUGG (m⁶A) CUGUG-3′) (SEQ ID NO: 15) was added to bind with YTHDF1 (361-559), and unbiotinylated m⁶A-containing mRNA was used as the positive control. Before Alpha signal detection, streptavidin donor beads and anti-His acceptor beads were added into the white assay plate in subdued light (OptiPlate™-384, PerkinElmer) and incubated at 4° C. for 1 hour to ensure sufficient binding between biotin tag and streptavidin donor beads, as well as that between His tag and anti-His acceptor beads. Then the Alpha signal was detected on Envision Readers (PerkinElmer).

As for competition assay, compound (e.g., SAA or SAC) was diluted in the same way, and non-biotinylated m⁶A-containing mRNA was diluted to 400 nM, 200 nM, 50 nM and 25 nM with assay buffer. 200 nM His-YTHDF1 (361-559) was incubated with non-biotinylated m⁶A-containing mRNA at 4° C. for 10 minutes. Then, the compound (e.g., SAA or SAC) was added in and incubated at room temperature for another 30 minutes in order to compete with m⁶A-containing mRNA to bind with the protein. Next, 20 nM biotinylated m⁶A-containing mRNA and two kinds of beads were added in subdued light and incubated at 4° C. for 1 hour before detection.

NMR Assay

NMR CPMG experiment was performed at 25° C. using Bruker Avance III spectrometer (600 MHz proton frequency) with a cryogenically cooled probe (Bruker biospin, Germany). YTHDF1 (361-559) was diluted to 20 μM, 10 μM and 5 μM in phosphate buffer (20 mM NaH₂PO₄, 20 mM Na₂HPO₄, 150 mM NaCl, pH 7.4, D₂O). The compound (e.g., SAA or SAC) was dissolved in 5% deuterated DMSO to the concentration of 200 μM. The solvent-suppressed 1D ¹H CPMG was obtained via the pulse sequence (RD-90°-(τ-180°-τ) n-ACQ). In the pre-saturation procedure, a 54.78 dB pulse in 4 s duration of the recycle delay (RD) was applied to eliminate water resonance. Then, the 900 pulse length was modulated to 11.82 μs approximately. And at last, a total of 4 dummy scans and 64 free induction decays (FIDs) were collected into 64000 acquisition points, covering a spectral width of 12 kHz (20 ppm) with the acquisition time (ACQ) of 2.73 s.

Isothermal Titration Calorimetry

Purified YTHDF1 (361-559) was dialyzed at 4° C. overnight in dialysis buffer (20 mM Hepes (pH 7.4) and 200 mM NaCl). Then dialyzed protein was diluted to 50 μM with dialysis buffer. The compound (e.g., SAA or SAC) was dissolved and diluted to 1 mM with dialysis buffer as well. Isothermal titration calorimetry (ITC) was conducted on a Microcal ITC 200 isothermal titration calorimeter (GE Healthcare) at 25° C. 200 μL 50 μM YTHDF1 (361-559) was filled in the sample cell and constantly stirred at 750 rpm; 40 μL 1 mM SAA was filled in the syringe. After one 0.4 μL injection, the compound (e.g., SAA or SAC) was first titrated into YTHDF1 (361-559) by nineteen 2 L injections at 180 s intervals. Then in order to make the titration effect more obvious, the nineteen 2 L injections were changed after calculation into five 2.5 μL injections followed by fourteen 1.9 μL injections. And 1 mM of compound (e.g., SAA or SAC) titrating into dialysis buffer was also performed as control to exclude the thermal effect of background dilution. The experimental data was analyzed via Microcal ORIGIN (v7.0) software (Microcal Software).

SPR Binding Assay

SPR binding assay was conducted at 25° C. on the Biacore T200 instrument (GE Healthcare). YTHDF1 (361-559) was covalently immobilized on a CM5 chip via standard amine-coupling procedure in the condition of 10 mM sodium acetate (pH 5.5). Then the compound (e.g., SAA or SAC) was diluted with HBS buffer (20 mM Hepes (pH 7.4), 200 mM NaCl and 0.4% (v/v) DMSO) in a gradient manner. Next, diluted compound (e.g., SAA or SAC) was injected to bind with the immobilized YTHDF1 (361-559) at the flow rate of 30 L/min for 60 s, followed by injection of HBS buffer for 600 s dissociation at the same flow rate. The equilibrium dissociation constant (K_{d}) values of the compound (e.g., SAA or SAC) was generated from data analysis with Biacore T200 evaluation software (GE Healthcare).

MST Assay

Microscale Thermophoresis (MST) assay was performed on MiscroScale Thermophoresis instrument (NanoTemper Technologies) at room temperature using the label-free method. The compound (e.g., SAA or SAC) was diluted from 1 mM in a 2-fold gradient manner with MST buffer (20 mM Hepes (pH 7.4), 200 mM NaCl and 0.1 mM Pluronic® F-127). And the YTHDF1 (361-559) was diluted to 4 μM with MST buffer. Then, 10 μL of the compound (e.g., SAA or SAC) and 10 μL YTHDF1 (361-559) were mixed together and incubated at room temperature for 20 minutes. Before measuring, the mixture was centrifuged at 13000 rpm for 10 minutes at 4° C. At last, the samples were collected by Monolith NT™ Automated Label Free capillaries (NanoTemper Technologies) and the measurement was started. The K_{d} value of the compound (e.g., SAA or SAC) was obtained from data analysis using MO. Affinity Analysis Software v2.3 (NanoTemper Technologies).

Hydrogen Deuterium Exchange Mass Spectrometry

YTHDF1 (361-559) was incubated with the compound (e.g., SAA) at 4° C. overnight before the measurement. In hydrogen deuterium exchange mass spectrometry (HDX MS), the hydrogen atoms of YTHDF1 (361-559)-apo and YTHDF1 (361-559)-SAA were exchanged with deuterium for 0 s, 10 s, 30 s, 60 s, 1200 s, 3600 s and 14400 s respectively at 10° C. in buffer containing 20 mM Hepes (pH 7.4), 200 mM NaCl and D₂O. And the reactions were stopped at 0.5° C. using buffer containing 4 M guanidine hydrochloride, 0.5 M TCEP and 100 mM citric acid (pH 2.3). After deuterium label reaction, the samples were digested at 4° C. by pepsin immobilized on the column in order to obtain peptides of the samples. These peptides were next separated by HPLC and analyzed via mass spectrometry, respectively. HDX MS data was analyzed in HDExaminer software (v2.4.1) and +5% were set as the threshold to pick out those peptides with significant change.

Cellular Thermal Shift Assay

The cellular thermal shift assay (CETSA) was conducted according to the protocol reported previously. 293T cell line (ATCC) used in the assay was cultured at 37° C., 5% CO₂, in DMEM culture medium (Life Technologies) containing 10% fetal calf serum (Gibco, U.S.A.), 1% penicillin/streptomycin (Life Technologies). 293T cells were collected after incubated with PBS buffer as control or 100 μM SAA for 4 hours and equally divided into twelve parts, respectively. The divided parts were heated under series of temperature range from 39° C. to 59° C. for 3 minutes and cooled at 4° C. for 3 minutes immediately. Then, the cells were lysed via freeze-thawing with liquid nitrogen and the protein samples were collected by centrifugation. Though adding in SDS loading and boiling at 99° C. for 5 minutes, the samples were prepared for western blot detection. In the western blot assays, GAPDH was used as the internal reference. And the quantitative analysis of the results of the assay was performed via ImageJ software.

Cell Line and Mice

B16-OVA is an OVA-transfected clone derived from the mouse melanoma cell line B16, was provided by Y.-X. Fu (UT Southwestern).

E.G7-OVA is an OVA-transfected clone derived from the murine thymic lymphoma cell line E.G7, was provided by Chen Dong (Tsinghua University).

Ythdf1^{-/+} mice were generated in house by the inventors as described in previous studies (e.g., see Shi, H. et al., Nature 563, 249−253 (2018)).

Ythdf1^{F/F} mice were provided by Bin Shen (Nanjing Medical University) and CD11c^{cre} mice were purchased from Jackson laboratory.

OT-I mice were ovalbumin specific CD8⁺ TCR transgenic mice, and were provided by Xiaohuan Guo (Tsinghua University).

Tumor Inoculation and Treatment

For B6 mice tumor growth, 5×10⁵ B16-OVA or 1×10⁶ E.G7-OVA tumor cells were inoculated subcutaneously (s.c.) into the flank of mice. Tumor length (a) and width (b) was measured every two days and tumor volume was calculated by the formula ab²/2. For the inhibitor treatment, 10 μM SAA or DMSO was injected intraperitoneally (i.p.) on day 9 and 11 after tumor inoculation. For other mice model (rag1^{-/-} and Ythdf1 conditional knockout), times and dose of inhibitor treatment was the same. For α-PD-L1 and SAA combination treatment, 5×10⁵ B16-OVA tumor cells were inoculated subcutaneously into the flank of mice. 100 μg α-PD-L1 antibody (clone 10F.9G2) or rat immunoglobulin were administered at day 9 after tumor inoculation. 10 μM SAA or DMSO was administered in the same way on day 9 and 11 after tumor injection.

FLT3L-DC Cultures and Inhibitor Treatment

Bone marrow was isolated from wild-type and Ythdf1^{-/-} mice and treated with red cell lysis buffer to remove red blood cell. IMDM medium containing 10% fetal bovine serum was used to suspend bone marrow cells. To culture FLT3L-DCs, cell concentration was adjusted to 1×10⁶/mL. Cells were cultured with 100 ng/mL FLT3L for 9 days to get mature FLT3L-DCs. Mature FLT3L-DCs were purified via EasySep Mouse CD11c Positive Selection Kit II, then treated with 10 μM SAA or DMSO in IMDM medium (contain 10% bovine and 100 ng/mL FLT3L) for 10 hours.

DC Antigen-Presentation Function Assay

For in vitro cross-presentation study, mature FLT3L-DCs were harvested on day 9 and purified by EasySep Mouse CD11 c Positive Selection Kit II, then treated with 10 μM SAA or DMSO in IMDM medium (contain 10% bovine and 100 ng/mL FLT3L) for 10 h. After inhibitor (e.g., SAA or SAC) treatment, FLT3L-DCs were co-cultured with necrotic B16-OVA cells for 6 hours. Then antigen-obtained DCs were purified and co-cultured with naïve T cells from OT-1 mice at a ratio of 1:10 for 96 h. The co-culture medium was 1640 RPMI contains 10% fetal bovine with or without 1 μg/mL OT-1 (OVA 257-264) peptide. For ex vivo DCs cross-presentation assay, four types of DCs (migration CD11b DC, migration CD103′ DC, resident CD11b DC and resident CD8⁺ DC) were sorted in draining lymph node from SAA treated B16-OVA bearing mice at day 12. These DCs were co-cultured with OT-1 naïve T cells in the ratio of 1:10 for 96 hours with or without OT-1 peptide. Supernatant IFN-γ production was detected via CBA assay.

T Cell Function Analysis

Tumor infiltrating leukocytes were resuspended by RPMI 1640 medium at 5×10⁶ per mL in 96-well plates. Phorbol-12-myristate-13-acetate (PMA) (2.5 μg/mL) and ionomycin (0.5 μg/mL) were used to stimulate T cells together with brefeldin A were added to the culture medium for 2 hours at 37° C. Cell concentration of total lymphocytes from draining lymph node was adjusted to 5×10⁶ per mL in 96-well plate. 1 μg/mL OT-1 (OVA 257-264) peptide was added to the wells and stimulated for 96 hours. Samples were stained with CD45 and CD8 on ice for 30 min. Intracellular staining was performed to quantify IFN-γ and granzyme B production.

Flow Cytometry

For flow cytometry analysis and DCs sorting, tumors, draining lymph nodes were gathered from mice and digested with 100 U/mL collagenase IV and 20 μg/mL DNase I at 37° C. for 40 min. Digestion was stopped by FACS buffer (PBS contains 2% FBS and 1 mM EDTA) and samples were filtered through 70-μm cell strainer. Samples were stained by specific antibodies in FACS buffer on ice for 30 min. Antibody information was described in Table 1 below. All samples were washed with FACS buffer after staining, cells were analyzed on BD Fortessa and sorted by AriaIII.

TABLE 1

| Antibody | Source | Catalog Number |
|---|---|---|
| Anti-mouse CD45 | Biolegend | 103138 |
| Anti-mouse CD4 | Biolegend | 100545 |
| Anti-mouse CD8 | Biolegend | 100714 |
| Anti-mouse NK1.1 | Biolegend | 108710 |
| Anti-mouse CD44 | Biolegend | 103025 |
| Anti-mouse CD62L | Biolegend | 104438 |
| Anti-mouse PD-1 | Biolegend | 135231 |
| Anti-mouse Tim-3 | Biolegend | 119717 |
| Anti-mouse CXCR5 | Biolegend | 145519 |
| Anti-mouse IFN-γ | Biolegend | 505808 |
| Anti-mouse Granzyme B | Biolegend | 515408 |

CFSE labelling

1×10⁷ lymphocytes form lymph nodes of naïve OT-I mice were washed with PBS twice, then resuspend in 1 mL PBS. 1 μL CFSE Tracker was added into suspension and incubated in 37° C. for 5 min. Then 5 mL RPMI-1640 medium with 10% FBS was added to stop CFSE labelling, incubated at room temperature for 5 min. After centrifugation, CFSE-labeled T cells were suspended with another 5 mL RPMI-1640 medium at least for 10 min in room temperature.

### Example 1 Inhibitory Activity Against YTHDF1

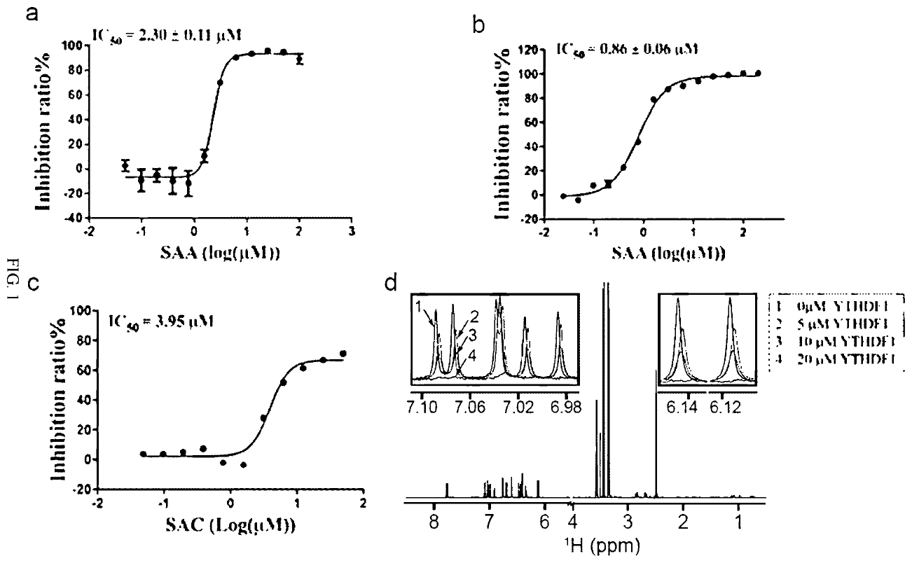

A high throughput screening (HTS) approach based on fluorescence polarization (FP) assay was developed to discover novel inhibitors of YTHDF1, and Salvianolic Acid A (SAA) was found to be a potent hit. The activity of SAA to inhibit the interaction between m⁶A-containing mRNA and YTHDF1 was evaluated in the FP assay, as shown in FIG. 1a, the IC₅₀ value obtained was 2.30±0.11 μM. To further validate the inhibitory activity of SAA, an AlphaScreen-based assay was performed, and as shown in FIG. 1b, the IC₅₀ value obtained was 0.86±0.06 μM, confirming that SAA could effectively block the binding between m⁶A and YTHDF1.

The inhibition of YTHDF1 activity with another salvianolic acid, Salvianolic Acid C (SAC), was also evaluated with FP assay, and as shown in FIG. 1c, the IC₅₀ value obtained was 3.95 μM.

Then, qualitative and quantitative experiments were performed to explore the binding between SAA and YTHDF1. Firstly, nuclear magnetic resonance (NMR) Carr-Purcell-Meiboom-Gill (CPMG) experiments were conducted. The results are shown in FIG. 1d, after adding 200 μM of SAA, a signal of the compound was detected in the CPMG spectrum, and the signal decreased when 5 μM, 10 μM and 20 μM YTHDF1 was added respectively, indicating a direct binding between SAA and YTHDF1.

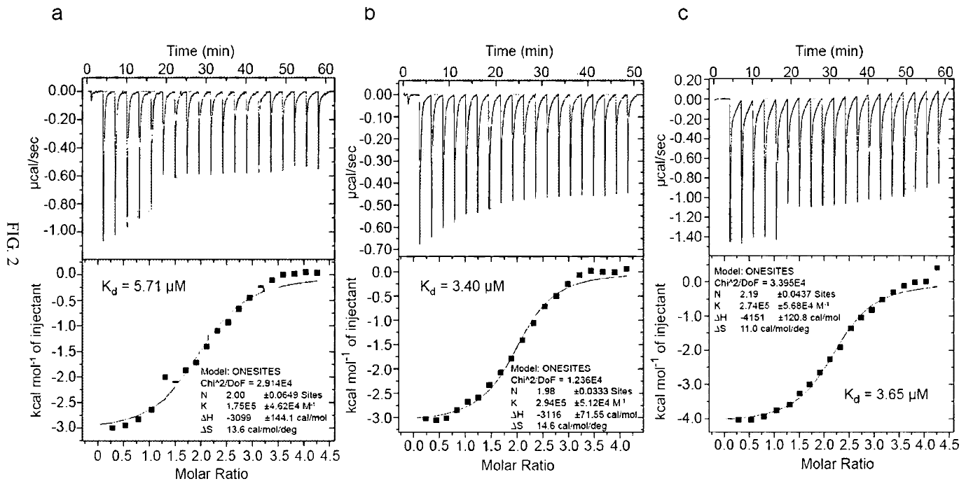

Then, the binding affinity between SAA and YTHDF1 was evaluated. The Isothermal Titration Calorimetry (ITC) assay was performed to accurately test the equilibrium dissociation constants (K_{d}) between YTHDF1 and SAA. The ITC assay was conducted on a Microcal iTC200 isothermal titration calorimeter (GE Healthcare) for three times independently. Briefly, freshly purified YTHDF1 (50 μM) was titrated with 1 mM SAA at 25° C. in a buffer comprising 20 mM Hepes (pH 7.4) and 200 mM NaCl. As shown in FIGS. 2a-2c, SAA bounds to YTHDF1 with a K_{d} value of 5.71 μM, confirming the binding between SAA and YTHDF1. Furthermore, the enthalpy change (ΔH=−3099+144.1 cal/mol) was less than zero which indicated that SAA could form hydrogen bond interactions with YTHDF1. In addition, the entropy change (ΔS=13.6 cal/mol/deg) was more than zero, indicating that the binding of SAA might induce conformational change of YTHDF1.

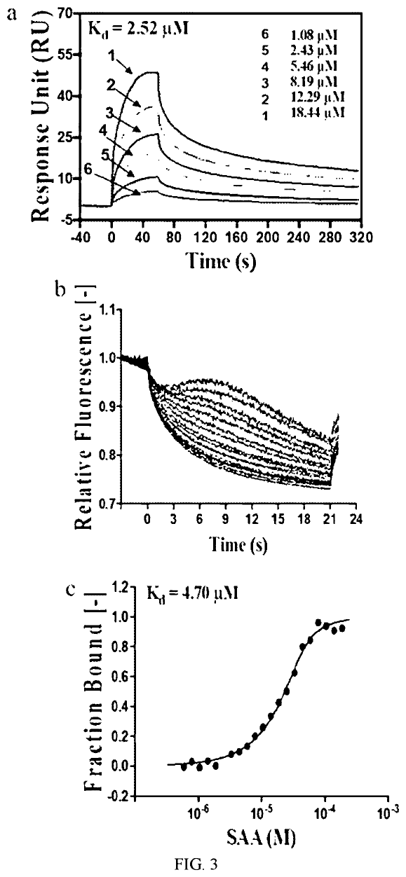

Further, surface plasmon resonance (SPR) assay and microscale thermophoresis (MST) assay were performed to confirm the binding strength between SAA and YTHDF1. As shown in FIGS. 3a-3c, a K_{d} value of 2.52 μM was obtained from SPR assay and a K_{d} value of 4.70 μM was obtained from MST assay, these results are consistent with that of the ITC experiments.

Taken together, it can be concluded that the compounds of the present application (e.g., SAA, SAC and other compounds of the present application) could bind to YTHDF1 directly and block its m⁶A binding activity in vitro.

### Example 2 Inhibiting YTHDF1 Activity Non-Competitively

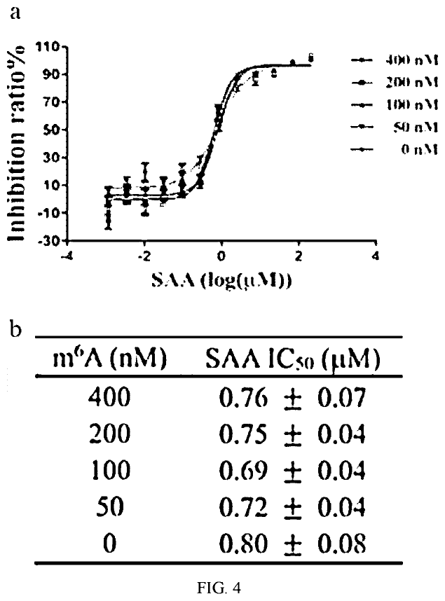

A competitive binding experiment was performed against fixed concentrations of non-biotinylated m⁶A-containing mRNA, based on AlphaScreen assay, using 200 nM YTHDF1 and 20 nM biotinylated m⁶A-containing mRNA. The results are shown in FIGS. 4a-4b, the inhibitory activity of SAA against YTHDF1 was 0.80±0.08 μM in the absence of non-biotinylated m⁶A-containing mRNA, and it remained unchanged when 50 nM, 100 nM, 200 nM or 400 nM non-biotinylated m⁶A-containing mRNA was preincubated with YTHDF1. These results demonstrated that SAA inhibits the functions of YTHDF1, but it doesn't compete with m⁶A for binding to YTHDF1.

Then, hydrogen deuterium exchange mass spectrometry (HDX MS) experiment was conducted, to determine the binding sites of SAA on YTHDF1. In the HDX MS assay, the HDX behaviors of YTHDF1-APO and YTHDF1-SAA were examined at 10 s, 30 s, 60 s, 1200 s, 3600 s and 14400 s and the changes of deuterium uptake between them were exhibited in the residue plot (FIG. 5a), the butterfly plot (FIG. 5b) and the heat map (FIG. 6) analysis. Many peptides possessed HDX percentage variation over 5%, indicating that the binding of SAA with YTHDF1 could induce significant conformational changes of the intact protein structure.

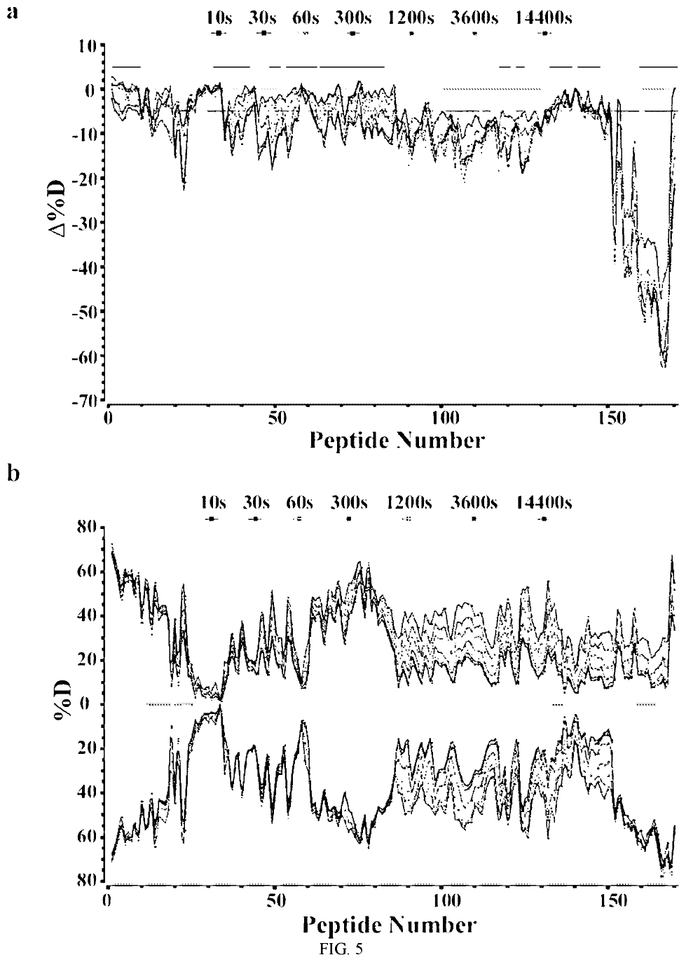

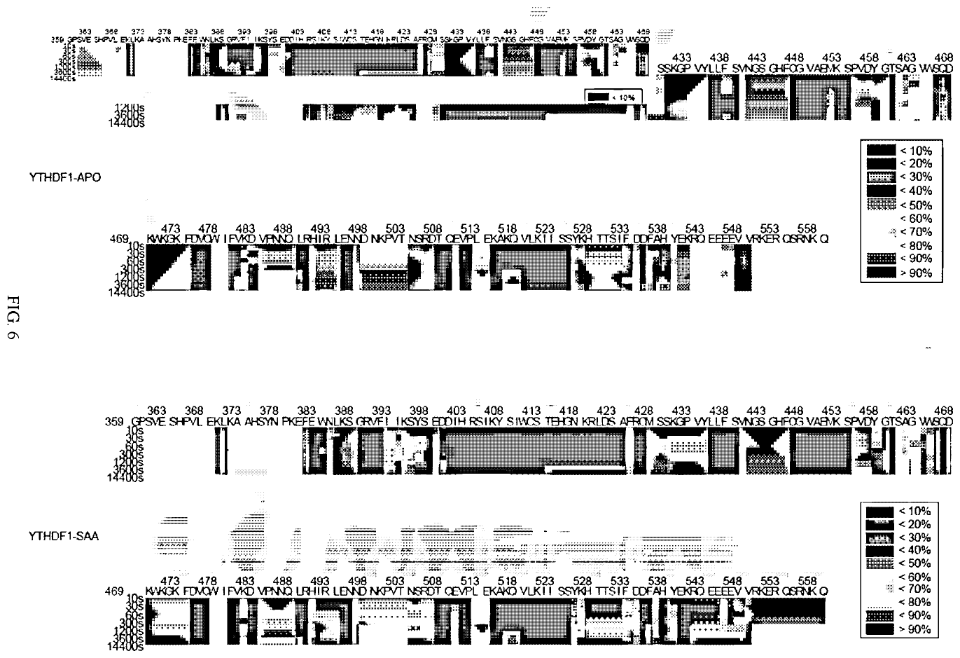

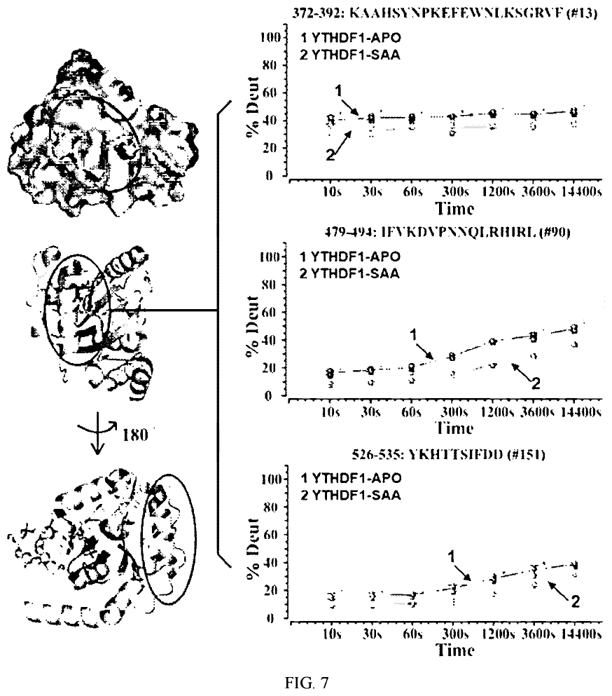

As shown in FIG. 7 and FIGS. 8a-8i, there were remarkable structural changes of YTHDF1 and relevant peptides, and the conformational change mainly occurred in the following three areas: 1) the m⁶A binding pocket, 2) a long and shallow pocket with certain positively charged amino acids, and 3) the C-terminal α-helix of YTHDF1 (which underwent the most significant change). These results suggest that SAA bind to YTHDF1 in one of these three areas and consequently induced conformational changes.

Then, YTHDF1 mutants and C-terminal truncates were designed (with amino acid sequences as set forth in SEQ ID NOs: 4-13), and these mutants and truncates were used to test the inhibitory activity of SAA.

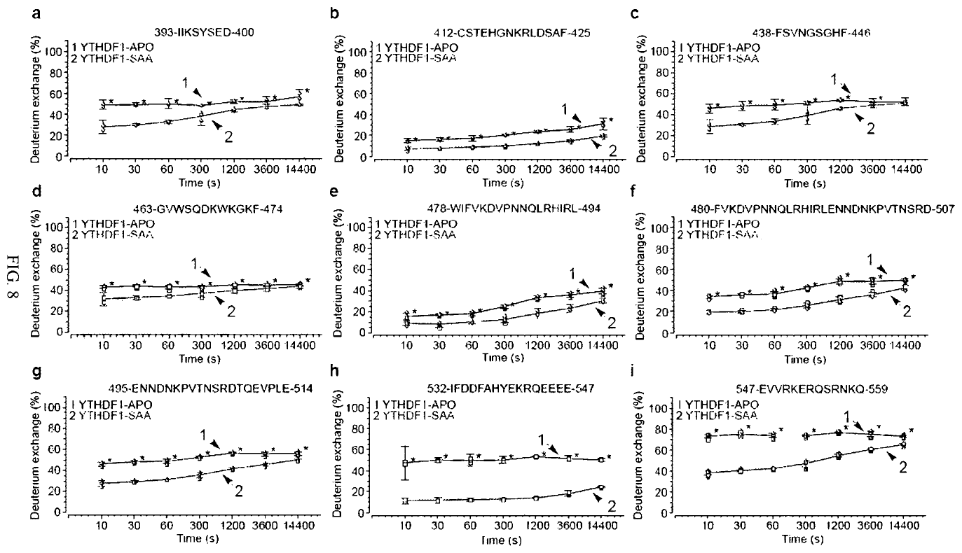

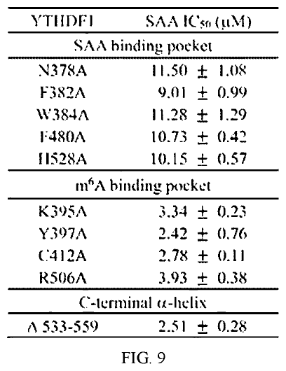

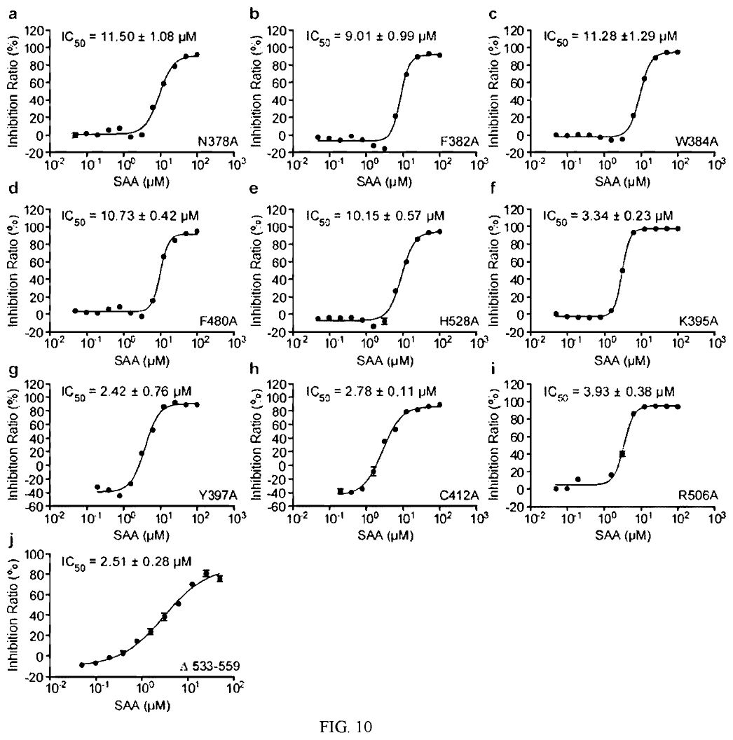

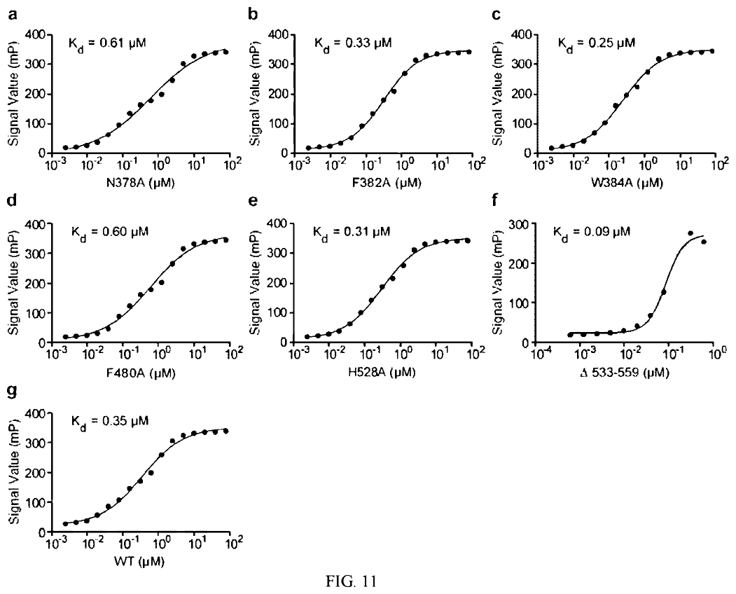

As shown in FIG. 9 and FIGS. 10f-10j, when the truncate (SEQ ID NO: 13) or the mutants with mutations in the m⁶A binding pocket (e.g., mutants with the following mutations K395A (SEQ ID NO: 9), Y397A (SEQ ID NO: 10), C412A (SEQ ID NO: 11), or R506A (SEQ ID NO: 12)) were used, the inhibitory activities of SAA against their binding to m⁶A were similar to that observed for the wildtype YTHDF1, as measured in the FP experiment. These results indicate that the C-terminal α-helix region and the m⁶A binding pocket are not essential for SAA binding. In contrast, as shown in FIG. 9 and FIGS. 10a-10e, when mutants (SEQ ID NOs: 4-8) with one or more mutations (e.g., mutation W384A, H528A, N378A, F480A or F382A) in residues 372-392, 479-494 or 526-535 were used, the inhibitory activities of SAA against their binding to m⁶A were significantly weaker than that against wildtype YTHDF1. And further investigation by FP assay showed that these mutations in residues 372-392, 479-494 or 526-535 as well as the C-terminal truncate did not influence the binding affinity between YTHDF1 and m⁶A (FIGS. 11a-11g).

Accordingly, it was found that SAA exerted non-competitive inhibitory activity against YTHDF1 through an allosteric mechanism, and at least partially via the hydrogen bond interactions formed between one or more residues within residues 372-392, 479-494 or 526-535 and SAA.

### Example 3 Binding of SAA to YTHDF1 in 293T

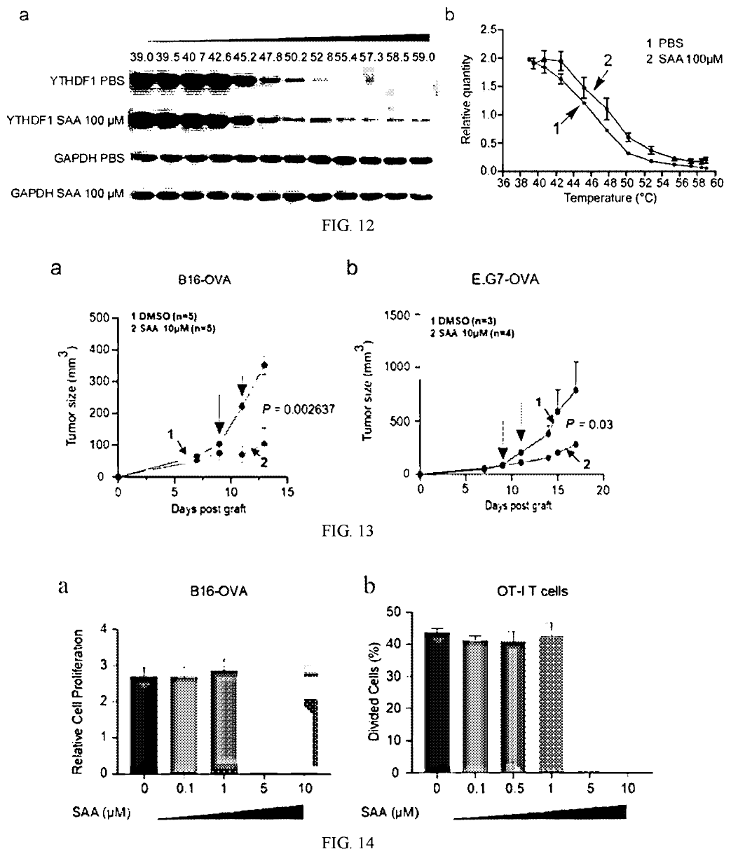

Further, the binding of SAA to YTHDF1 in cells was also examined. Cellular thermal shift (CETSA) assay was performed with 293T cell line, which was collected for heating at temperatures as indicated. After incubating the cells with 100 μM SAA for 4 hours, YTHDF1 protein in the incubated 293T cells were examined with western blot. As shown in FIGS. 12a-12b, the stability of YTHDF1 was improved after incubating with SAA and the curve shifted for about 2 degrees to the right, confirming that SAA could directly bind to YTHDF1 in the cytoplasm.

### Example 4 Inhibition of Tumor Growth In Vivo

5×10⁵ Ovalbumin (OVA)-expressing B16 melanoma cells were inoculated subcutaneously into wild-type mice. Tumor bearing mice were subsequently treated with 10 μM SAA 9 days and 11 days after tumor inoculation, and tumor growth was monitored. It was observed that tumor growth was much slower in mice receiving SAA in comparison with control group, as shown in FIG. 13a.

In another experiment, wild-type mice were injected subcutaneously with 1×10⁶ E.G7-OVA cells. 10 μM SAA was then injected to each mouse on day 9 and day 11. Tumor growth was monitored. A similar tendency of inhibition was found in E.G7-OVA lymphoma model (FIG. 13b).

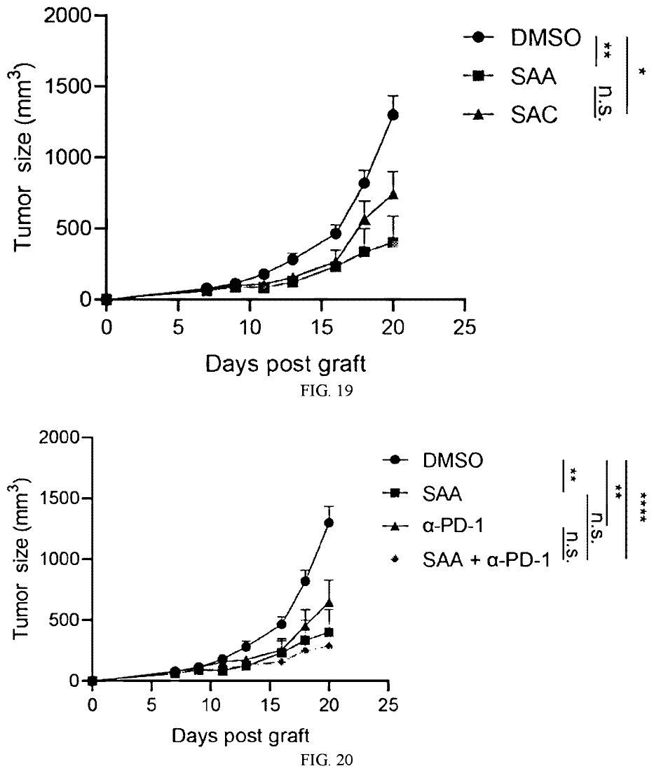

As shown in FIG. 19, 5×10⁵ B16-OVA cells were subcutaneously inoculated into C57BL/6 mice (n=17). 9 days after implantation, the mice were divided into three groups by their tumor size. DMSO (n=6), 10 μM SAA (n=6) or 10 μM SAC (n=5) was i.v. injected respectively. Tumor growth was monitored. Data are shown as mean±s.e.m., and “n.s.” means no significance, “*” p<0.05, “**” p<0.01 by unpaired one-tail t-test. It was observed that tumor growth was much slower in mice receiving SAA or SAC.

### Example 5 Effects on Tumor Cell Proliferation In Vitro

In a further experiment, tumor cells were treated with SAA in vitro. Briefly, 5×10⁴ B16-OVA tumor cells were treated with SAA of various doses respectively in a 96-well plate. Cell number was counted 12 hours after said SAA treatment. It was found that the proliferation of tumor cells was not affected even with increasing doses of SAA (FIG. 14a). These results suggest that SAA does not exert its anti-tumor effects by directly killing tumor cells.

### Example 6 Adaptive Immunity is Required for SAA Anti-Tumor Activity

In a further experiment, 5×10⁵ CFSE-labeled OT-I T cells were treated with SAA of various doses in 96-well plates, then the cells were stimulated with 1 μg/mL OT-I peptide for 24 hours. Divided T cells were analyzed by FACS. As shown in FIG. 14b, SAA could not influence T cell proliferation in vitro.

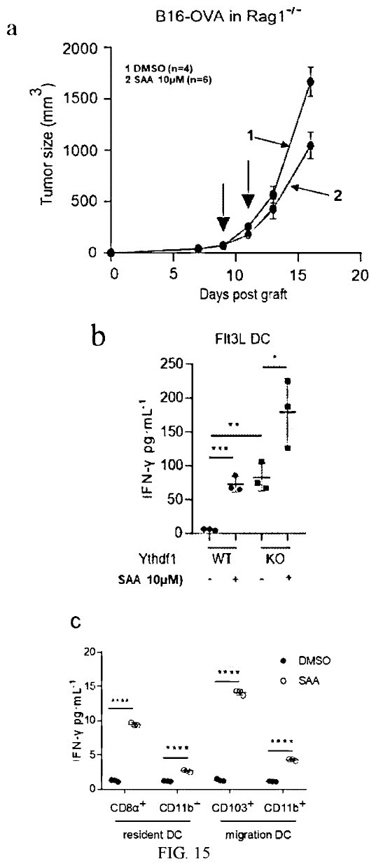

T/B cell-deficient Rag1^{-/-} mice were inoculated with 1×10⁵ B16-OVA cells, and 10 μM SAA was then injected to each mouse from day 7 to day 9, and tumor growth was monitored, wild type mice were used as control group. As shown in FIG. 15a, tumor growth was arrested in wild-type mice, but not in T/B cell-deficient Rag1^{-/-} mice, indicating that adaptive immunity is required for the maximal anti-tumor therapeutic effect of SAA.

### Example 7 SAA Enhances Cross-Presentation Functions of APCs

Bone marrow derived cells (wildtype or Ythdf1 gene deficient) were cultured with FLT3L for 9 days to get FLT3L-DC and these DCs were treated with 10 μM SAA for 12 hours, then, FLT3L-DCs were co-cultured with necrotic B16-OVA tumor cell for 6 hours. CD11c+ cells were purified and co-cultured with OT-I T cells for 72 hours. IFN-γ production was assessed by IFN-γ cytometric bead array.

As shown in FIG. 15b, FLT3L-DCs treated with SAA exhibited better ability in cross-priming T cells than control group. To compare the efficacy of SAA treatment with that of Ythdf1 gene knock out, Ythdf1 gene deficient DC (obtained from Ythdf1^{-/-} bone marrow) was used as positive control. Interestingly, SAA could also promote the cross-presentation function of Ythdf1^{-/-} FLT3L-DCs. These results indicate that the compounds of the present application (e.g., SAA) could promote the cross-priming functions of antigen presenting cells (e.g., DCs) in vitro.

Further, four types of classical DCs (resident CD11b⁺, resident CD8α⁺, migratory CD11b⁺ and migratory CD103⁺) in draining lymph node were sorted from SAA treated B16-OVA bearing wild-type mice on day 12. These DCs were co-cultured with OT-I T cells for 72 hours. Then, IFN-γ production was assessed by IFN-γ cytometric bead array. As shown in FIG. 15c, all these DCs subtypes from SAA treated mice showed better cross-priming of T cells than DMSO treated group, especially resident CD8^{α}+DC and migration CD103 DC. These results indicate that the compounds of the present application (e.g., SAA) could also promote the cross-priming functions of antigen presenting cells (e.g., DCs) in vivo.

### Example 8 SAA Functions in APCs to Strengthen Immune Response

Dendritic cells can activate T cells through cross-priming and/or direct-priming. In direct-priming process, DCs could stimulate T cells via surface co-stimulatory molecules such as CD80/CD86 or cytokines related with T cell activation. To evaluate whether the direct priming function was also affected after SAA administration, bone marrow derived cells (wildtype or Ythdf1 gene deficient) were cultured with FLT3L for 9 days to get FLT3L-DC and these DCs were treated with 10 μM SAA for 12 hours, then, FLT3L-DCs were co-cultured with necrotic B16-OVA tumor cell for 6 hours. CD11 c+ cells were purified and co-cultured with OT-I T cells with the addition of 1 μg/mL OT-I peptide for 72 hours. IFN-γ production was assessed by IFN-γ cytometric bead array. Further, four types of classical DCs (resident CD11b⁺, resident CD8α⁺, migratory CD11b⁺ and migratory CD103⁺) in draining lymph node were sorted from SAA treated B16-OVA bearing wild-type mice on day 12. These DCs were co-cultured with OT-I T cells with the addition of 1 μg/mL OT-I peptide for 72 hours. Then, IFN-γ production was assessed by IFN-γ cytometric bead array.

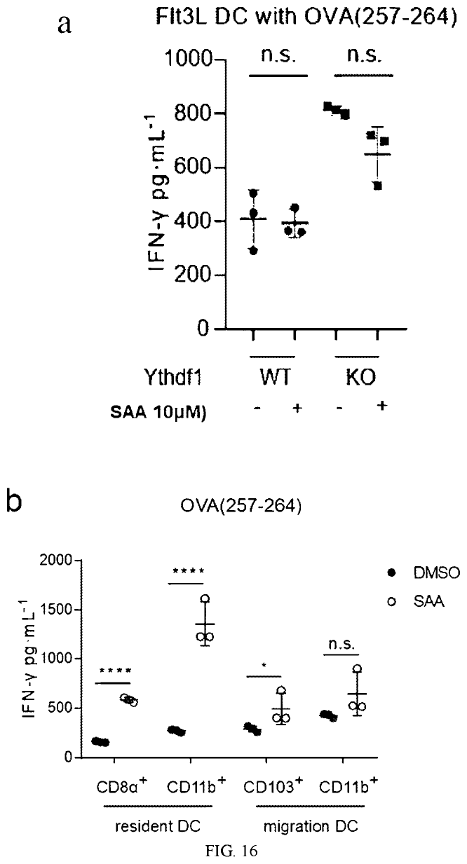

As shown in FIGS. 16a-16b, although SAA could not enhance FLT3L-DC direct-priming function in vitro, 3 of the 4 DC subtypes showed improved direct-priming capacity after SAA treatment, indicating SAA could enhance direct-priming capacity of DCs in vivo. These results suggest that the compounds of the present application (e.g., SAA) could function in APCs (e.g., dendritic cells) to strengthen immune responses.

### Example 9 APCs are the Major Targets of SAA

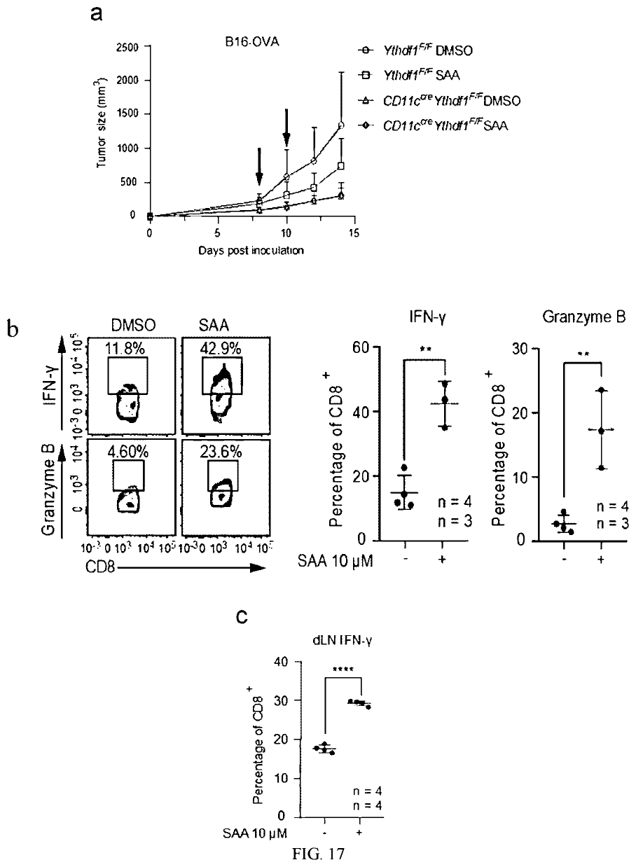

To determine whether DCs are the major targets of SAA, Ythdf1^{F/F} and CD11c^{cre}Ythdf1^{F/F} mice were injected subcutaneously with 2×10⁶ B16-OVA cells. Then, 10 μM SAA was injected to each mouse on day 9 and day 11, and tumor growth was monitored. As shown in FIG. 17a, The tumor growth in SAA treated Ythdf1^{F/F} mice showed analogous situation as observed in CD11c^{cre}Ythdf1^{F/F} mice, however, no further evident effect of tumor control was found in CD11c^{cre}Ythdf1^{F/F} mice, indicating that DCs are the major target of SAA.

### Example 10 SAA Activated Tumor Specific T Cells

To explore the effects of SAA treatment on T cell function, SAA treated B16-OVA bearing mice were sacrificed to investigate tumor infiltrating T cell (TILs) function. Firstly, Phorbol-12-myristate-13-acetate (PMA) and ionomycin were used to nonspecifically stimulate tumor infiltrating T cell, then intracellular staining was performed to quantify cytokine (e.g., IFN-γ, granzyme B) production by FACS. As shown in FIG. 17b, tumor infiltrating T cells from SAA treated mice secreted higher level of cytokines than DMSO group, indicating that SAA could strengthen tumor infiltrating T cell effector function.

Then, lymphocytes from draining lymph node were isolated and stimulated with 1 μg/mL OT1 peptide, and IFN-γ producing cell were analyzed by FACS. As shown in FIG. 17c, DLN T cells from SAA treated mice produced much more IFN-γ than DMSO group. The results demonstrated that there are more tumor specific T cells activated in draining lymph nodes after SAA administration.

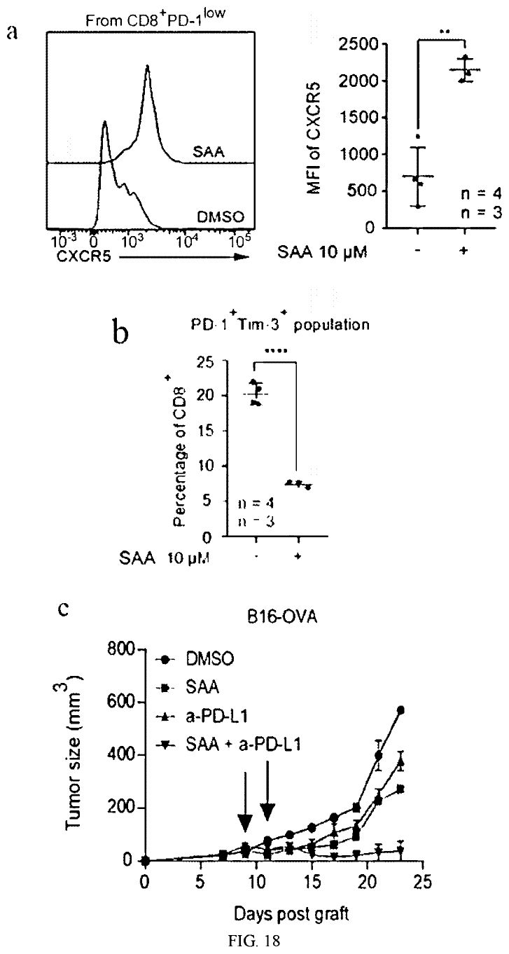

In addition, it was discovered that PD-1^{low} population from SAA treated group expressed much more CXCR5 than DMSO group (FIG. 18a), there PD-1^{low}CXCR5^{high} T cells (progenitor exhausted T cells) were detected 14 days after tumor inoculation with SAA treatment. In addition, terminally exhausted T cells (PD-1 and Tim-3 double positive cells) were assessed 14 days after tumor inoculation with SAA treatment, and it was observed that the frequency of these tumor infiltrating terminally exhausted T cells (PD-1⁺Tim-3⁺) decreased in SAA treated mice (FIG. 18b), confirming the enhanced anti-tumor activity.

These results show that T cells were better primed in the draining lymph nodes and the effector function of TILs were significantly improved after SAA treatment.

### Example 11 Synergistic Effects in Combination with Immune Checkpoint Inhibitors

Wild-type mice were injected subcutaneously with 5×10⁵ B16-OVA cells. 10 μM SAA was injected to each mouse on day 9 and day 11. Mice were also treated with 100 ug anti-PD-L1 antibody on day 9. Tumor growth was monitored over time. As shown in FIG. 18c, SAA or anti-PD-L1 antibody single treatment could partially inhibit B16-OVA tumor growth while the combination treatment could suppress tumor dramatically, even achieving complete tumor regressions. These results further support that SAA could induce improved T cell anti-tumor ability through enhanced DC function, and immune checkpoint inhibitors (such as anti-PD-L1 antibodies) can provoke more durable T cell response in combination with the compounds of the present application (e.g. SAA).

As shown in FIG. 20, 5×10⁵ B16-OVA cells were subcutaneously inoculated into C57BL/6 mice (n=23). 9 days after implantation, the mice were divided into two groups by their tumor size. At day 9 and 11 after implantation, one group was i.v treated with 10 μM SAA (n=6) while another was DMSO (n=6). Each of the two groups were further separated into another two groups at day 12 after inoculation, then i.p. injected with 200 μg PD-1 blockade antibody (Bio-X-cell, BE0146, clone: RMP1-14). Control group was PBS and DMSO treatment, n=6. Single treatment group was injected isotype control with SAA or α-PD-1 with DMSO, n=6. Combination group was treated with SAA and PD-1 blockade antibody, n=5. Tumor growth was monitored. Data are shown as mean±s.e.m., and “n.s.” means no significance, “**” p<0.01, “****” p<0.00001 by unpaired one-tail t-test. The results further support that immune checkpoint inhibitors (such as anti-PD-1 antibodies) can provoke more durable T cell response in combination with the compounds of the present application (e.g. SAA).

### Example 12 SAA Functions in APCs to Exhibit Durable Anti-Tumor Function

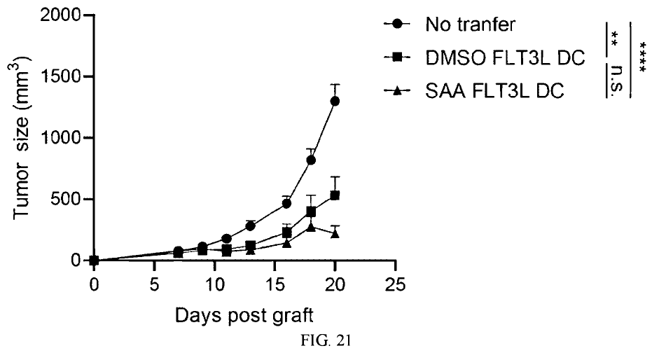

As shown in FIG. 21, pre-treat mature FLT3L DC with DMSO or 10 μM SAA for 10 h, then co-culture these DCs with necrotic B16-OVA for 6 h. CD11c⁺ cells were purified for the adoptive transfer. 5×10⁵ B16-OVA cells were subcutaneously inoculated into C57BL/6 mice (n=17). 9 days after implantation, the mice were divided into three groups by their tumor size. 1×10⁵ DMSO-treated FLT3L DCs (n=6) or SAA-treated FLT3L DCs (n=5) were i.v. transferred into the tumor bearing mice. 7 days after first transfer, second batch of adoptive transfer was performed. Tumor growth was monitored. Data are shown as mean s.e.m., and “n.s.” means no significance, “**” p<0.01, “****” p<0.00001 by unpaired one-tail t-test. The results support that adoptive transfer FLT3L DC, treated with the compounds of the present application (e.g. SAA), exhibits durable anti-tumor function.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A YTH N6-Methyladenosine RNA Binding Protein 1 (YTHDFT) attenuating agent, wherein the agent comprises a compound, and when bound to YTHDF1, the compound binds to at least one residue selected from the group consisting of amino acid residues 372-392, 479-494 and 526-535 of SEQ ID NO: 1.

2. The YTHDF1 attenuating agent of claim 1, wherein, when bound to YTHDF1, the compound binds to at least one residue selected from the group consisting of N378, F382, W384, F480, and H528 of SEQ ID NO: 1.

3-4. (canceled)

5. The YTHDF1 attenuating agent of any of claim 1, wherein the compound comprises at least one compound selected from the group consisting of Formula I, a prodrug, a metabolite, a derivative of a compound of Formula I, a pharmaceutically acceptable salt, a pharmaceutically acceptable ester, and a pharmaceutically acceptable amide; 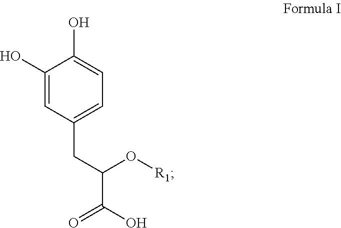
wherein R₁ is at least one selected from the group consisting of C₁₋₅₀ hydrocarbyl, C₁₋₅₀ substituted hydrocarbyl, C₁₋₅₀ heterohydrocarbyl and C₁₋₅₀ substituted heterohydrocarbyl.

6-15. (canceled)

16. The YTHDF1 attenuating agent of claim 1, wherein the compound comprises at least one compound selected from the group consisting of a prodrug, a metabolite, a derivative of any of the following compounds, a pharmaceutically acceptable salt, a pharmaceutically acceptable ester, a pharmaceutically acceptable amide,

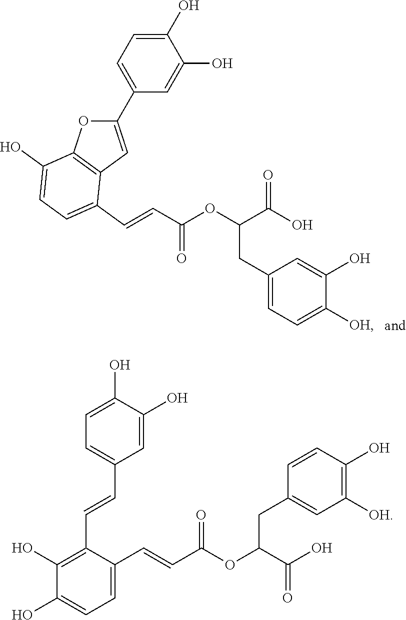 [SMILES: O=C(/C=C/c1ccc(O)c2oc(-c3ccc(O)c(O)c3)cc12)OC(Cc1ccc(O)c(O)c1)C(=O)O ; "and" ; O=C(/C=C/c1ccc(O)c(O)c1/C=C/c1ccc(O)c(O)c1)OC(Cc1ccc(O)c(O)c1)C(=O)O]

17-20. (canceled)

21. A modified antigen presenting cell (mAPC), wherein the mAPC has been treated with the YTHDF1 attenuating agent of claim 1.

22. (canceled)

23. A composition, comprising the YTHDF1 attenuating agent of claim 1, and/or a mAPC treated with the YTHDF1 attenuating agent, and optionally a pharmaceutically acceptable carrier.

24-27. (canceled)

28. The composition of claim 23, further comprising a second active ingredient, wherein the second active ingredient is an anti-cancer agent, and wherein the second active ingredient comprises at least one agent selected from the group consisting of an anti-PD-L1 antibody or an antigen binding portion thereof, an anti-PD-1 antibody or an antigen binding portion thereof, an anti-CTLA-4 antibody or an antigen binding portion thereof, and an IDO inhibitor.

29. (canceled)

30. The composition of claim 23, further comprising a second active ingredient, wherein the second active ingredient is an anti-cancer agent, and wherein the second active ingredient is capable of causing an increase of tumor antigens in a subject receiving the composition.

31-32. (canceled)

33. A method for attenuating an activity of YTHDF1, comprising:
administering an effective amount of the YTHDF1 attenuating agent of claim 1.

34. (canceled)

35. A method for determining whether or not a candidate agent is a YTHDF1 attenuating agent, comprising:
contacting the candidate agent with a YTHDF1 mutant, wherein the YTHDF1 mutant comprises an amino acid substitution, deletion or addition of at least one residue selected from the group consisting of residues 372-392, 479-494 and 526-535 of SEQ ID NO: 1.

36-37. (canceled)

38. A kit, comprising a YTHDF1 mutant.

39. A method for manufacturing a compound with a YTHDF1 attenuating agent;
wherein, when bound to YTHDF1, the compound binds to at least one residue selected from the group consisting of amino acid residues 372-392, 479-494 and 526-535 of SEQ ID NO: 1.

40-53. (canceled)

54. The method of claim 39, wherein the compound comprises at least one compound, selected from the group consisting of a prodrug, a metabolite, a derivative of any of the following compounds, a pharmaceutically acceptable salt, a pharmaceutically adaptable ester, a pharmaceutically acceptable amide,

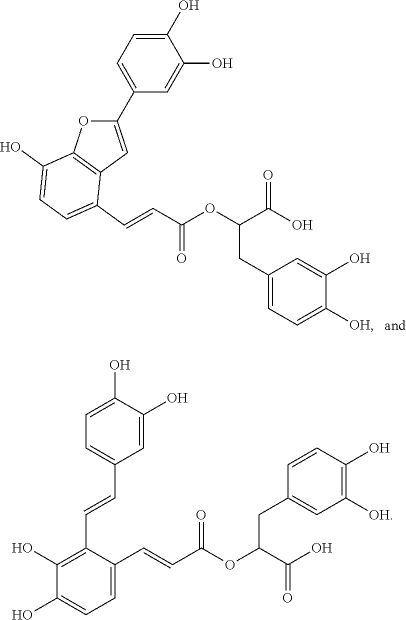 [SMILES: O=C(/C=C/c1ccc(O)c2oc(-c3ccc(O)c(O)c3)cc12)OC(Cc1ccc(O)c(O)c1)C(=O)O ; "and" ; O=C(/C=C/c1ccc(O)c(O)c1/C=C/c1ccc(O)c(O)c1)OC(Cc1ccc(O)c(O)c1)C(=O)O]

55-58. (canceled)

59. A method for activating an APC or a DC, wherein the method comprises:
administering the YTHDF1 attenuating agent of claim 1 to the APC.

60. (canceled)

61. A method for treating a disease, disorder or condition associated with an expression of an antigen in a subject in need thereof, for treating cancer in a subject in need thereof, for stimulating a T cell-mediated immune response to a cancer cell and/or a tumor antigen in a subject in need thereof, for providing an anti-tumor immunity in a subject in need thereof, for preventing and/or reversing exhaustion of T cells in a subject in need thereof, and/or for enhancing an activity of T cells in a subject in need thereof, comprising,
administering to the subject:
the YTHDF1 attenuating agent of claim 1;
a mAPC treated with the YTHDF1 attenuating agent; and
a composition comprising the YTHDF1 attenuating agent and the mAPC.

62-79. (canceled)

80. The method of claim 61, wherein the subject has received, is receiving, or will receive an anti-cancer treatment.

81-86. (canceled)

87. The method of claim 61, wherein the method further comprises:
administering to the subject an additional anti-cancer treatment.

88-89. (canceled)

90. The method of claim 87, wherein the additional anti-cancer treatment comprises at least one agent selected from the group consisting of an anti-PD-L1 antibody or an antigen binding portion thereof, an anti-PD-1 antibody or an antigen binding portion thereof, an anti-CTLA-4 antibody or an antigen binding portion thereof, and an IDO inhibitor.

91. (canceled)

92. The method of claim 87, wherein the additional anti-cancer treatment is capable of causing an increase of tumor antigens in the subject.

93. (canceled)

94. A method, comprising:
administering the YTHDF1 attenuating agent of claim 1, a mAPC treated with the YTHDF1 attenuating agent, and a composition comprising the YTHDF1 attenuating agent and the mAPC to a subject in need thereof; and
1) activating an APC;
2) activating a DC;
3) generating an immune cell having enhanced anti-tumor activity;
4) preventing or reversing exhaustion of an immune cell;
5) treating a disease, disorder or condition associated with an expression of an antigen in a subject in need thereof;
6) treating cancer in the subject in need thereof;
7) stimulating an immune cell mediated immune response to a cancer cell or a tumor antigen in the subject in need thereof;
8) providing an anti-tumor immunity in the subject in need thereof;
9) increasing or improving proliferation and/e or activity of immune cells;
10) increasing or improving proliferation or activity of tumor specific immune cells;
11) enhancing cytokine production of T cells;
12) enhancing an antitumor response of a cancer immunotherapy; and
13) inhibiting tumor growth, inhibiting proliferation of tumor cells, or killing tumor cells.

95-103. (canceled) thereof, an anti-PD-1 antibody or an antigen binding portion thereof, an anti-CTLA-4 antibody or an antigen binding portion thereof, and an IDO inhibitor.

101. The use of any of claims 97-100, wherein said additional active ingredient comprises pembrolizumab, nivolumab, cemiplimab, atezolizumab, avelumab, durvalumab, ipilimumab, and/or an antigen binding fragment or a derivative of any of the foregoing.

102. The use of any of claims 97-101, wherein said additional active ingredient is capable of causing an increase of one or more tumor antigens in a subject receiving it.

103. The use of claim 102, wherein said tumor antigen is selected from the group consisting of: CEA, gp100, the MAGE family of proteins, DAGE, GAGE, RAGE, NY-ESO 1, Melan-A/MART 1, TRP-1, TRP-2, tyrosinase, HER-2/neu, MUC-1, p53, KSA, PSA, PSMA, and fragments and modified versions thereof.
